(19) United States
(12) Patent Application Publication
(10) Pub. No.: US 2025/0311972 A1
(43) Pub. Date: Oct. 9, 2025

(54) GRAPHENE SENSOR SYSTEMS FOR DETECTION OF TOXIC COMPOUNDS AND/OR COMBINATIONS OF COMPOUNDS

(71) Applicant: VOCxI Health, Inc., Arden Hills, MN (US)

(72) Inventors: Gregory J. Sherwood, White Bear Lake, MN (US); Raia Colette Finc, Shoreview, MN (US); Raj Vaibhav Gude, Minneapolis, MN (US)

(21) Appl. No.: 19/172,146

(22) Filed: Apr. 7, 2025

Related U.S. Application Data
(60) Provisional application No. 63/631,129, filed on Apr. 8, 2024.
(60) Provisional application No. 63/687,010, filed on Aug. 26, 2024.

(51) Int. Cl. A61B 5/00; A61B 5/08

(52) U.S. Cl. CPC A61B 5/4845; A61B 5/082

(57) ABSTRACT

Embodiments herein relate to systems and methods for detecting poisoning of an individual with a toxic compound such as a cyanide compound and detecting overdoses with compounds or combinations of compounds. In an embodiment, a system herein includes a sensor device that can include a set of graphene sensor elements. At least some of the set of graphene sensor elements are modified with metalloporphyrin compounds. The system can also include a measurement circuit. The system can be configured to receive a breath sample of the subject, take measurements of the breath sample using the set of graphene sensor elements to generate sample data, and evaluate the sample data to detect a pattern showing an enhanced response of the set of graphene sensor elements modified with metalloporphyrins indicating poisoning of the subject and/or the presence of an overdosed compound or combinations of compounds. Other embodiments are also included herein.

This application claims the benefit of U.S. Provisional Application No. 63/631,129, filed Apr. 8, 2024, and U.S. Provisional Application No. 63/687,010, filed Aug. 26, 2024, the contents of which are herein incorporated by reference in their entirety.

### FIELD

Embodiments herein relate to systems and methods for detecting poisoning of an individual with a compound such as a cyanide compound, as well as the presence of compounds such as fentanyl, and/or combinations of compounds.

### BACKGROUND

Cyanide poisoning is a serious and potentially fatal condition that occurs when cyanide, a fast-acting and potent chemical, enters the body. Cyanide can be found in various forms, including gas, liquid, and solid forms. Common sources of cyanide exposure include industrial processes such as metal cleaning and electroplating, manufacturing of paper, textiles, and plastics, and the use of chemicals in photography. Additionally, cyanide can be found in smoke from house fires. Certain plants and foods naturally contain cyanide, particularly in seeds and pits of some fruits.

Cyanide acts by inhibiting a crucial enzyme in the body's cells. This enzyme, cytochrome c oxidase, is essential for the process of oxidative phosphorylation, which is a critical component of cellular respiration. Cyanide binds to the iron within this enzyme and thereby disrupts the electron transport chain, preventing cells from utilizing oxygen to produce adenosine triphosphate (ATP). Without ATP, cells cannot function, leading to the failure of organs and ultimately, if untreated, death.

Symptoms of cyanide poisoning can appear rapidly after exposure and may include headache, dizziness, confusion, weakness, and shortness of breath, leading to seizures, loss of consciousness, and cardiac arrest in severe cases. Immediate medical attention is crucial for anyone with cyanide poisoning. However, the symptoms of cyanide poisoning can easily be confused with other conditions and thus diagnosing cyanide poisoning can be extremely difficult within a relevant time frame for appropriate treatment.

Fentanyl is an extremely potent synthetic opioid. Fentanyl is 50 to 100 times more potent than morphine and heroin and even a small amount can be lethal. Use of fentanyl can lead to respiratory depression, which can lead to respiratory arrest and death if not treated promptly. Illegally made fentanyl is often mixed with other drugs making them more potent and dangerous. Many users may not be aware of what they are ingesting.

### SUMMARY

Embodiments herein relate to systems and methods for detecting poisoning of an individual with a toxic compound such as a cyanide compound, fentanyl, combinations of compounds, or the like. In a first aspect, a system for detecting poisoning of a subject can be included having a sensor device. The sensor device can include a set of graphene sensor elements, wherein at least some of the set of graphene sensor elements can be modified with metalloporphyrin compounds. The system can also include a measurement circuit. The system can be configured to receive a breath sample of the subject, take measurements of the breath sample using the set of graphene sensor elements to generate sample data, and evaluate the sample data to detect a pattern showing an enhanced response of the set of graphene sensor elements modified with metalloporphyrins indicating poisoning of the subject.

In a second aspect, in addition to one or more of the preceding or following aspects, or in the alternative to some aspects, the metalloporphyrin compounds can include cobalt metalloporphyrins.

In a third aspect, in addition to one or more of the preceding or following aspects, or in the alternative to some aspects, the metalloporphyrin compounds can include iron metalloporphyrins.

In a fourth aspect, in addition to one or more of the preceding or following aspects, or in the alternative to some aspects, the measurement circuit can be configured to provide a voltage stimulus to the sensor device and measure resulting capacitance values of the graphene sensor elements.

In a fifth aspect, in addition to one or more of the preceding or following aspects, or in the alternative to some aspects, the voltage stimulus can be provided over a range of voltage values.

In a sixth aspect, in addition to one or more of the preceding or following aspects, or in the alternative to some aspects, the set of graphene sensor elements can include graphene varactors.

In a seventh aspect, in addition to one or more of the preceding or following aspects, or in the alternative to some aspects, the system can be a system for detecting poisoning with a cyanide anion.

In an eighth aspect, in addition to one or more of the preceding or following aspects, or in the alternative to some aspects, the system can be a system for detecting poisoning with fentanyl or mustard gas.

In a ninth aspect, in addition to one or more of the preceding or following aspects, or in the alternative to some aspects, the enhanced response includes one or more of a change in the Dirac point upon a forward voltage sweep, a change in the Dirac point upon a reverse voltage sweep, a change in the capacitance of the Dirac point, and a change in the hysteresis between the forward and reverse Dirac points.

In a tenth aspect, in addition to one or more of the preceding or following aspects, or in the alternative to some aspects, the enhanced response includes a change in the minimum capacitance of the Dirac point.

In an eleventh aspect, in addition to one or more of the preceding or following aspects, or in the alternative to some aspects, the enhanced response can be relative to a non-metalloporphyrin modified graphene sensor.

In a twelfth aspect, a system for detecting poisoning of a subject can be included having a sensor device. The sensor device can include a set of graphene sensor elements, wherein one or more compounds can be disposed on a surface of the set of graphene sensor elements and the one or more compounds can include metalloporphyrins. The system can also include a measurement circuit. The system can be configured to receive a breath sample of the subject, take measurements of the breath sample using the set of graphene sensor elements to generate sample data, and evaluate the sample data to detect an enhanced response of the set of graphene sensor elements with metalloporphyrins disposed thereon indicating poisoning of the subject.

In a thirteenth aspect, in addition to one or more of the preceding or following aspects, or in the alternative to some aspects, the metalloporphyrins can include cobalt metalloporphyrins.

In a fourteenth aspect, in addition to one or more of the preceding or following aspects, or in the alternative to some aspects, the metalloporphyrins can include iron metalloporphyrins.

In a fifteenth aspect, in addition to one or more of the preceding or following aspects, or in the alternative to some aspects, the one or more compounds can be in the form of a self-assembling monolayer.

In a sixteenth aspect, in addition to one or more of the preceding or following aspects, or in the alternative to some aspects, the measurement circuit can be configured to provide a voltage stimulus to the sensor device and measure resulting capacitance values of the graphene sensor elements.

In a seventeenth aspect, in addition to one or more of the preceding or following aspects, or in the alternative to some aspects, the voltage stimulus can be provided over a range of voltage values.

In an eighteenth aspect, in addition to one or more of the preceding or following aspects, or in the alternative to some aspects, the set of graphene sensor elements can include graphene varactors.

In a nineteenth aspect, in addition to one or more of the preceding or following aspects, or in the alternative to some aspects, the system can be a system for detecting poisoning with a cyanide anion.

In a twentieth aspect, in addition to one or more of the preceding or following aspects, or in the alternative to some aspects, the system can be a system for detecting poisoning with fentanyl or mustard gas.

In a twenty-first aspect, in addition to one or more of the preceding or following aspects, or in the alternative to some aspects, the enhanced response includes one or more of a change in the Dirac point upon a forward voltage sweep, a change in the

Dirac point upon a reverse voltage sweep, a change in the capacitance of the Dirac point, and a change in the hysteresis between the forward and reverse Dirac points.

In a twenty-second aspect, in addition to one or more of the preceding or following aspects, or in the alternative to some aspects, the enhanced response includes a change in the minimum capacitance of the Dirac point.

In a twenty-third aspect, in addition to one or more of the preceding or following aspects, or in the alternative to some aspects, the enhanced response can be relative to a non-metalloporphyrin modified graphene sensor.

In a twenty-fourth aspect, a method of detecting poisoning of a subject can be included. The method can include taking a breath sample of the subject, taking measurements of the breath sample using a graphene sensor modified with one or more metalloporphyrins to generate sample data, and evaluating the sample data to detect a response associated with metalloporphyrin modified graphene.

In a twenty-fifth aspect, in addition to one or more of the preceding or following aspects, or in the alternative to some aspects, the metalloporphyrins can be in the form of a self-assembling monolayer.

In a twenty-sixth aspect, in addition to one or more of the preceding or following aspects, or in the alternative to some aspects, the method can further include providing a voltage stimulus to the graphene sensor and measuring resulting capacitance values of the graphene sensor elements.

In a twenty-seventh aspect, in addition to one or more of the preceding or following aspects, or in the alternative to some aspects, the method can further include providing a voltage stimulus to the graphene sensor over a range of voltage values and measuring resulting capacitance values of the graphene sensor elements.

In a twenty-eighth aspect, in addition to one or more of the preceding or following aspects, or in the alternative to some aspects, the graphene sensor includes a graphene varactor.

In a twenty-ninth aspect, in addition to one or more of the preceding or following aspects, or in the alternative to some aspects, the method can be a method of detecting poisoning with a cyanide anion.

In a thirtieth aspect, in addition to one or more of the preceding or following aspects, or in the alternative to some aspects, the method can be a method for detecting poisoning with fentanyl or mustard gas.

In a thirty-first aspect, in addition to one or more of the preceding or following aspects, or in the alternative to some aspects, the response associated with metalloporphyrin modified graphene includes one or more of a change in the Dirac point upon a forward voltage sweep, a change in the Dirac point upon a reverse voltage sweep, a change in the capacitance of the Dirac point, and a change in the hysteresis between the forward and reverse Dirac points.

In a thirty-second aspect, in addition to one or more of the preceding or following aspects, or in the alternative to some aspects, the response associated with metalloporphyrin modified graphene includes a change in the minimum capacitance of the Dirac point.

In a thirty-third aspect, in addition to one or more of the preceding or following aspects, or in the alternative to some aspects, the response associated with metalloporphyrin modified graphene can be relative to a non-metalloporphyrin modified graphene sensor.

In a thirty-fourth aspect, a system for detecting poisoning of a subject can be included having a sensor device. The sensor device can include a set of graphene sensor elements, wherein at least some of the set of graphene sensor elements can be modified with metalloporphyrin compounds and/or other compounds, and a measurement circuit. The system can be configured to receive a breath sample of the subject, take measurements of the breath sample using the set of graphene sensor elements to generate sample data, and evaluate the sample data to detect a pattern showing an enhanced response of the set of graphene sensor elements modified with metalloporphyrins and/or one or more other modifying compounds indicating the presence of a particular compound or a particular combination of compounds in the subject.

In a thirty-fifth aspect, in addition to one or more of the preceding or following aspects, or in the alternative to some aspects, the metalloporphyrin compounds can include a ruthenium metalloporphyrin.

In a thirty-sixth aspect, in addition to one or more of the preceding or following aspects, or in the alternative to some aspects, the measurement circuit can be configured to provide a voltage stimulus to the sensor device and measure resulting capacitance values of the graphene sensor elements.

In a thirty-seventh aspect, in addition to one or more of the preceding or following aspects, or in the alternative to some aspects, the voltage stimulus can be provided over a range of voltage values.

In a thirty-eighth aspect, in addition to one or more of the preceding or following aspects, or in the alternative to some aspects, the set of graphene sensor elements can include graphene varactors.

In a thirty-ninth aspect, in addition to one or more of the preceding or following aspects, or in the alternative to some aspects, the system can be a system for detecting the presence of and/or overdose with fentanyl.

In a fortieth aspect, in addition to one or more of the preceding or following aspects, or in the alternative to some aspects, the system can be a system for detecting the presence of and/or overdose with fentanyl and another compound.

In a forty-first aspect, in addition to one or more of the preceding or following aspects, or in the alternative to some aspects, the system can be a system for distinguishing between overdose with fentanyl alone and overdose with fentanyl and another compound.

In a forty-second aspect, in addition to one or more of the preceding or following aspects, or in the alternative to some aspects, the system can be a system for distinguishing between overdose with fentanyl alone and overdose with fentanyl and ketamine and/or xylazine.

In a forty-third aspect, in addition to one or more of the preceding or following aspects, or in the alternative to some aspects, the enhanced response includes one or more of a change in the Dirac point upon a forward voltage sweep, a change in the Dirac point upon a reverse voltage sweep, a change in the capacitance of the Dirac point, and a change in the hysteresis between the forward and reverse Dirac points.

In a forty-fourth aspect, in addition to one or more of the preceding or following aspects, or in the alternative to some aspects, the enhanced response includes a change in the minimum capacitance of the Dirac point.

In a forty-fifth aspect, in addition to one or more of the preceding or following aspects, or in the alternative to some aspects, the enhanced response can be relative to a non-metalloporphyrin modified graphene sensor.

In a forty-sixth aspect, a system for detecting poisoning of a subject can be included having a sensor device. The sensor device can include a set of graphene sensor elements, wherein one or more compounds can be disposed on a surface of the set of graphene sensor elements. The one or more compounds can include metalloporphyrins and/or other modifying compounds. The system can also include a measurement circuit. The system can be configured to receive a breath sample of the subject, take measurements of the breath sample using the set of graphene sensor elements to generate sample data, and evaluate the sample data to detect an enhanced response of the set of graphene sensor elements with metalloporphyrins and/or one or more other modifying compounds thereon indicating the presence of a particular compound or a particular combination of compounds in the subject.

In a forty-seventh aspect, in addition to one or more of the preceding or following aspects, or in the alternative to some aspects, the metalloporphyrins can include a ruthenium metalloporphyrin.

In a forty-eighth aspect, in addition to one or more of the preceding or following aspects, or in the alternative to some aspects, the one or more compounds can be in the form of a self-assembling monolayer.

In a forty-ninth aspect, in addition to one or more of the preceding or following aspects, or in the alternative to some aspects, the measurement circuit can be configured to provide a voltage stimulus to the sensor device and measure resulting capacitance values of the graphene sensor elements.

In a fiftieth aspect, in addition to one or more of the preceding or following aspects, or in the alternative to some aspects, the voltage stimulus can be provided over a range of voltage values.

In a fifty-first aspect, in addition to one or more of the preceding or following aspects, or in the alternative to some aspects, the set of graphene sensor elements can include graphene varactors.

In a fifty-second aspect, in addition to one or more of the preceding or following aspects, or in the alternative to some aspects, the system can be a system for detecting the presence of and/or overdose with fentanyl.

In a fifty-third aspect, in addition to one or more of the preceding or following aspects, or in the alternative to some aspects, the system can be a system for detecting the presence of and/or overdose with fentanyl and another compound.

In a fifty-fourth aspect, in addition to one or more of the preceding or following aspects, or in the alternative to some aspects, the system can be a system for distinguishing between overdose with fentanyl alone and overdose with fentanyl and another compound.

In a fifty-fifth aspect, in addition to one or more of the preceding or following aspects, or in the alternative to some aspects, the system can be a system for distinguishing between overdose with fentanyl alone and overdose with fentanyl and ketamine and/or xylazine.

In a fifty-sixth aspect, in addition to one or more of the preceding or following aspects, or in the alternative to some aspects, the enhanced response includes one or more of a change in the Dirac point upon a forward voltage sweep, a change in the Dirac point upon a reverse voltage sweep, a change in the capacitance of the Dirac point, and a change in the hysteresis between the forward and reverse Dirac points.

In a fifty-seventh aspect, in addition to one or more of the preceding or following aspects, or in the alternative to some aspects, the enhanced response includes a change in the minimum capacitance of the Dirac point.

In a fifty-eighth aspect, in addition to one or more of the preceding or following aspects, or in the alternative to some aspects, the enhanced response can be relative to a non-metalloporphyrin modified graphene sensor.

In a fifty-ninth aspect, a method of detecting poisoning of a subject can be included. The method can include taking a breath sample of the subject, taking measurements of the breath sample using a graphene sensor modified with one or more metalloporphyrins to generate sample data, and evaluating the sample data to detect a response associated with metalloporphyrin modified graphene and/or graphene modified with other compounds.

In a sixtieth aspect, in addition to one or more of the preceding or following aspects, or in the alternative to some aspects, the metalloporphyrins can be in the form of a self-assembling monolayer.

In a sixty-first aspect, in addition to one or more of the preceding or following aspects, or in the alternative to some aspects, the method can further include providing a voltage stimulus to the graphene sensor and measuring resulting capacitance values of the graphene sensor elements.

In a sixty-second aspect, in addition to one or more of the preceding or following aspects, or in the alternative to some aspects, the method can further include providing a voltage stimulus to the graphene sensor over a range of voltage values and measuring resulting capacitance values of the graphene sensor elements.

In a sixty-third aspect, in addition to one or more of the preceding or following aspects, or in the alternative to some aspects, the graphene sensor includes a graphene varactor.

In a sixty-fourth aspect, in addition to one or more of the preceding or following aspects, or in the alternative to some aspects, the method can be a method of detecting the presence of and/or overdose with fentanyl.

In a sixty-fifth aspect, in addition to one or more of the preceding or following aspects, or in the alternative to some aspects, the method can be a method for detecting the presence of and/or overdose with fentanyl and one or more other compounds.

In a sixty-sixth aspect, in addition to one or more of the preceding or following aspects, or in the alternative to some aspects, the method can be a method for distinguishing between overdose with fentanyl alone and overdose with fentanyl and another compound.

In a sixty-seventh aspect, in addition to one or more of the preceding or following aspects, or in the alternative to some aspects, the method can be a method for distinguishing between overdose with fentanyl alone and overdose with fentanyl and ketamine and/or xylazine.

In a sixty-eighth aspect, in addition to one or more of the preceding or following aspects, or in the alternative to some aspects, the response associated with metalloporphyrin modified graphene includes one or more of a change in the Dirac point upon a forward voltage sweep, a change in the Dirac point upon a reverse voltage sweep, a change in the capacitance of the Dirac point, and a change in the hysteresis between the forward and reverse Dirac points.

In a sixty-ninth aspect, in addition to one or more of the preceding or following aspects, or in the alternative to some aspects, the response associated with metalloporphyrin modified graphene includes a change in the minimum capacitance of the Dirac point.

In a seventieth aspect, in addition to one or more of the preceding or following aspects, or in the alternative to some aspects, the response associated with metalloporphyrin modified graphene can be relative to a non-metalloporphyrin modified graphene sensor.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of various components of a system in accordance with various embodiments herein.

FIG. 2 is a schematic top plan view of a chemical sensor element in accordance with various embodiments herein.

FIG. 3 is a schematic diagram of a portion of a measurement zone in accordance with various embodiments herein.

FIG. 4 is a formula of metalloporphryins used in accordance with various embodiments herein.

FIG. 5 is a formula of metalloporphryins used in accordance with various embodiments herein.

FIG. 6 is a formula of metalloporphryins used in accordance with various embodiments herein.

FIG. 7 is a formula of metalloporphryins used in accordance with various embodiments herein.

FIG. 8 is a formula of metalloporphryins used in accordance with various embodiments herein.

FIG. 9 is a formula of metalloporphryins used in accordance with various embodiments herein.

FIG. 10 is a formula of metalloporphryins used in accordance with various embodiments herein.

FIG. 11 is a formula of metalloporphryins used in accordance with various embodiments herein.

FIG. 12 is a formula of metalloporphryins used in accordance with various embodiments herein.

FIG. 13 is a formula of metalloporphryins used in accordance with various embodiments herein.

FIG. 14 is a formula of metalloporphryins used in accordance with various embodiments herein.

FIG. 15 is a formula of metalloporphryins used in accordance with various embodiments herein.

FIG. 16 is a formula of metalloporphryins used in accordance with various embodiments herein.

FIG. 17 is a formula of metalloporphryins used in accordance with various embodiments herein.

FIG. 18 is a formula of metalloporphryins used in accordance with various embodiments herein.

FIG. 19 is a formula of metalloporphryins used in accordance with various embodiments herein.

FIG. 20 is a schematic perspective view of a graphene varactor in accordance with various embodiments herein.

FIG. 21 is a schematic cross-sectional view of a portion of a graphene varactor in accordance with various embodiments herein.

FIG. 22 is a schematic diagram of a passive sensor circuit and a portion of a reading circuit in accordance with various aspects herein.

FIG. 23 is a schematic diagram of exemplary measurement circuity to measure the capacitance of a plurality of graphene sensors in accordance with various embodiments herein.

FIG. 24 is a set of charts showing the response of graphene varactors modified with various chemical compounds before and after exposure to a cyanide containing gas sample.

FIG. 25 is a set of charts showing the response of graphene varactors modified with various chemical compounds to fentanyl and other compounds.

FIG. 26 is a radar plot showing the results of fentanyl exposure in combination with other compounds.

While embodiments are susceptible to various modifications and alternative forms, specifics thereof have been shown by way of example and drawings and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Immediate medical attention is crucial for anyone in a poisoning or overdose situation (such as cyanide poisoning or a fentanyl overdose). However, the symptoms of cyanide poisoning (as one example of a poison) can easily be confused with other conditions and thus diagnosing poisoning can be extremely difficult within a relevant time frame for appropriate treatment. Similarly, the symptoms of overdoses of one compound can easily be confused with those of other compounds making diagnosis and appropriate treatment of overdoses difficult. Making treatment of overdoses even more difficult, other compounds may be mixed with compounds that may be overdosed (such as fentanyl) or otherwise taken at the same time, but proper treatment frequently depends on knowing what compounds are present.

However, systems herein can include chemical sensor elements that include sensitivity to various compounds including poisons and compounds that may be overdosed (compounds such as cyanide and/or cyanide anion, fentanyl, combinations of fentanyl with other compounds, etc.) and can be used to rapidly detect poisoning, overdoses, and the compounds causing the same. In some embodiments, chemical sensor elements herein can be configured to bind poison and/or overdosed compounds and combinations of compounds in a complex sample mixture, such as a gaseous sample mixture like a breath sample. The chemical sensor elements can specifically include graphene-based sensors. For example, the chemical sensor elements can include graphene-based quantum capacitance varactors (“graphene varactors”) that can exhibit a change in capacitance in response to an applied voltage as a result of the presence of one or more analytes, such as poisons and/or overdosed compounds on a surface of the graphene varactor. In this way, gas samples can be analyzed by contacting them with a graphene varactor-based sensor element, providing a bias voltage, and measuring capacitance or voltage values. In many cases, the graphene varactor-based sensor elements can be exposed to a range of bias voltages to discern features such as the Dirac point (or the bias voltage at which the varactor exhibits the lowest capacitance). The response signal generated by the measurement circuitry and can be used to characterize the content of the gaseous mixture allowing rapid and accurate identification of poisons, overdosed compounds, and/or combinations of compounds.

Graphene surfaces of graphene sensors herein can be modified with a layer of a compound, such as one or more metalloporphyrins or other surface modifying compounds that make the sensor particularly sensitive to some compounds. Also, some graphene surfaces can be bare or be modified with a layer of a compound that make them particularly insensitive to some compounds. As such, the presence of cyanide (as a specific example of a poison) or fentanyl (as a specific example of an overdosed compounds) or combinations of fentanyl and other compounds can be detected by observing a pattern including a response in those sensor components that are sensitive to a particular compound and/or a lesser response or a lack of a response in those sensor components that are not sensitive to the compounds or compounds. The sensors of embodiments herein can be used to rapidly detect cyanide in the breath (or another fluid sample) and determine that cyanide poisoning has taken place. In some embodiments, the sensors of embodiments herein can also be used to detect a likely time frame of cyanide exposure.

In some embodiments, the presence of other toxic or overdosed compounds can also be detected including, for example, toxic compounds that have electronegative atoms with a lone pair of electrons such as nitrogen or oxygen. In some embodiments, systems herein can be used to detect toxic compounds wherein the metal atom (cobalt, rhodium, etc.) of the metalloporphyrin catalyzes breakdown of the toxic compound. In some embodiments, systems herein can be used to detect toxic compounds including fentanyl and mustard gas. In various embodiments, systems herein can be used to distinguish between the presence of fentanyl alone versus fentanyl in combination with another compound.

Systems herein can be used to detect compounds in humans, but also in other animals, such any animal from with a gaseous sample (such as breath or another gaseous sample) can be taken. As such, systems herein can be used to detect compounds such as poisons, overdosed compounds, or other toxic compounds in various mammals, ruminants, canines, felines, and the like.

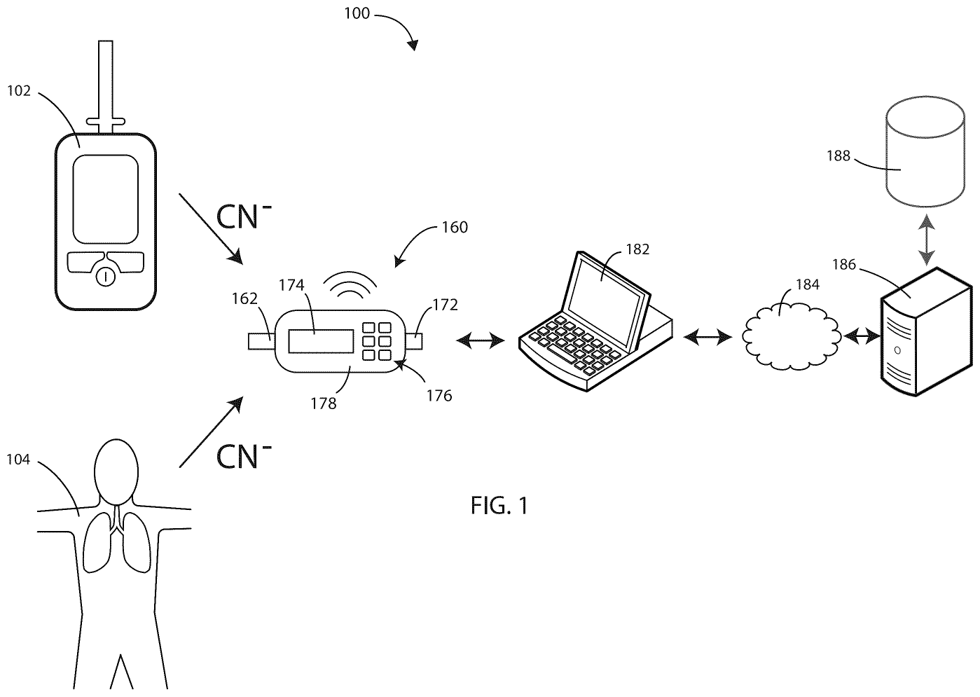

It will be appreciated that systems herein can take on various physical forms and include various components. Referring now to FIG. 1, a schematic view of a system 100 for measuring analyte presence in a gaseous sample is shown in accordance with various embodiments herein. The system 100 can include a sensing device 160 that includes a chemical sensor element with a plurality of discrete graphene components for sensing analytes in a gaseous mixture. The discrete graphene components can include graphene-based variable capacitors (or graphene varactors), as will be described in more detail below. The term “discrete graphene component” can include “graphene varactor” as a specific example thereof but can also include other graphene containing components such as graphene field-effect transistors and the like unless otherwise specified or the context dictates otherwise.

In the embodiment shown in FIG. 1, the sensing device 160 of system 100 is depicted in a hand-held format that can be used in the field, medical clinic, workplace, and the like. It will be appreciated that the system herein can further include a table-top system for use in a medical clinic, hospital, laboratory, etc. However, it will be appreciated that many other formats for the sensing device 160 and system 100 are also contemplated herein.

The sensing device 160 can include a housing 178 and an air intake port 162. In some embodiments, air intake port 162 can be in fluid communication with one or more separate gas sampling devices 102. In other embodiments, air intake port 162 can be configured as a mouthpiece into which a subject 104 to be evaluated can blow a breath sample. In yet other embodiments, the air intake port 162 can itself act as a gas sampling device. The sensing device 160 can be configured to actively draw a gas into housing 178 or it can be configured to receive a gas passively from a subject 104 or a gas sampling device 102. In some embodiments, the sensing device 160 can include a flow control valve in fluid communication with an upstream flow path relative the chemical sensor element. In various embodiments, the flow control valve can control fluid communication between an upstream flow path and the chemical sensor element.

The sensing device 160 can also include a display screen 174 and a user input device 176, such as a keyboard. The sensing device 160 can also include a gas outflow port 172. In some embodiments, the system 100 can include a local computing device 182 that can include a microprocessor, input and output circuits, input devices, a visual display, a user interface, and the like. In some embodiments, the sensing device 160 can communicate with the local computing device 182 to exchange data between the sensing device 160 and the local computing device 182. The local computing device 182 can be configured to perform various processing steps with the data received from the sensing device 160, including, but not limited to, calculating various parameters of the graphene varactors described herein. However, it should be appreciated that in some embodiments the features associated with the local computing device 182 can be integrated into the sensing device 160. In some embodiments, the local computing device 182 can be a laptop computer, a desktop computer, a server (real or virtual), a purpose dedicated computer device, or a portable computing device (including, but not limited to, a mobile phone, tablet, wearable device, etc.). The local computing device 182 and/or the sensing device 160 can communicate with computing devices in remote locations through a data network 184, such as the Internet or another network for the exchange of data as packets, frames, or otherwise.

In some cases, various operations can be performed to facilitate processing at the edge (e.g., with the sensing device 160 and/or a local computing device 182). By way of example, in some cases template patterns used herein, data for toxic compound (cyanide and others) patterns, or other data can be saved in memory by the sensing device 160 and/or a local computing device 182 for use in executing operations herein. In some embodiments, some data can be discarded or otherwise not used in order to simplify calculations herein. By way of example, in some embodiments, the system can be configured to drop, discard, or otherwise not utilize data relating to compounds other than those of relevance for detecting toxic compounds. In some embodiments, the system can be configured to drop, discard, or otherwise not utilize data relating to compounds other than cyclic and/or aromatic compounds.

However, in some embodiments, various operations herein can be performed, at least in part, by remote computing resources. For example, in some embodiments, the system 100 can also include a computing device such as a server 186 (real or virtual). In some embodiments, the server 186 can be located remotely from the sensing device 160. The server 186 can be in data communication with a database 188. The database 188 can be used to store various subject information, such as that described herein. In some embodiments, the database 188 can specifically include characteristic patterns of data (or templates) associated with the presence of cyanide or other toxic compounds at multiple different time points after ingestion (such as 30 seconds, 1, 2, 3, 4, 5, 10, 15, 20, or 30 minutes after ingestion, or longer, or an amount falling within a range between any of the foregoing), and the like.

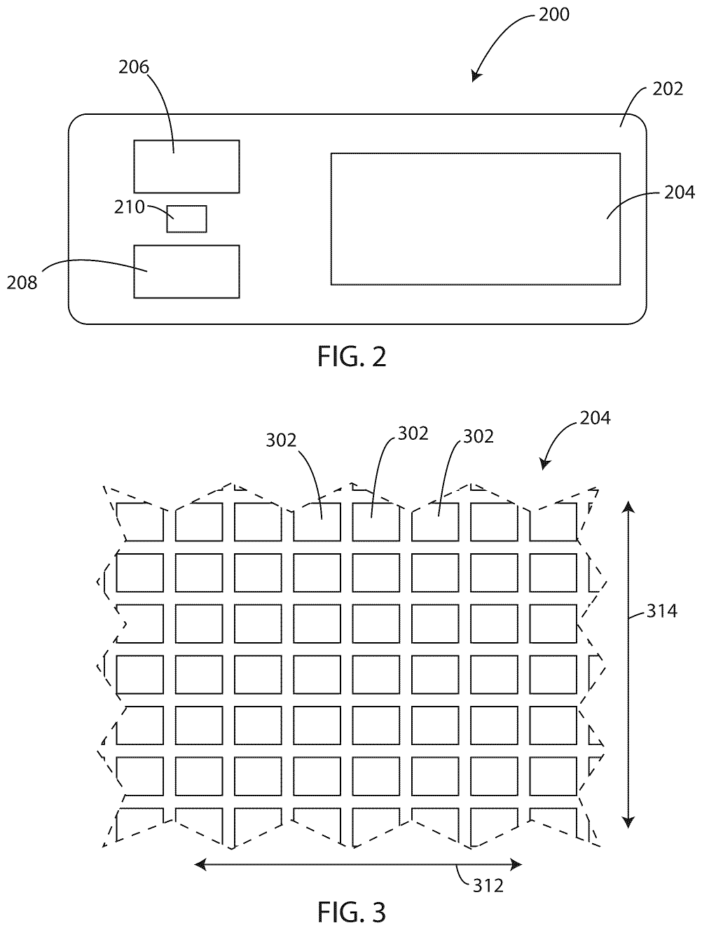

Referring now to FIG. 2, a schematic top plan view of a chemical sensor element 200 is shown in accordance with various embodiments herein. The chemical sensor element 200 can include a substrate 202. It will be appreciated that the substrate can be formed from many different materials, including silicon, glass, quartz, sapphire, polymers, metals, glasses, ceramics, cellulosic materials, composites, metal oxides, and the like. The thickness of the substrate can vary. In some embodiments, the substrate has sufficient structural integrity to be handled without undue flexure that could damage components thereon.

The chemical sensor elements herein can include a first measurement zone 204, and second measurement zone 206, and a third measurement zone 208 that can be disposed on the substrate 202. It will be appreciated that more than three measurement zones can be present on the chemical sensor elements herein. In some embodiments, the first measurement zone 204 can define at least a portion of a first gas flow path. The first measurement zone 204 can include a plurality of discrete graphene components that can sense analytes in a gaseous sample, such as a breath sample. The second measurement zone 206 can define at least a portion of a second gas flow path. In some embodiments, the second gas flow path can be entirely separate from the first gas flow path, while in other embodiments the second gas flow path can include a portion of the first gas flow path.

The second measurement zone 206 can also include a plurality of discrete graphene components. The chemical sensor element can include a component 210 to store reference data. The component 210 to store reference data can be an electronic data storage device, an optical data storage device, a printed data storage device (such as a printed code), or the like.

Each chemical sensor element herein can include one or more discrete graphene components in an array throughout the measurement zones. Referring now to FIG. 3, a schematic diagram of a portion of a chemical sensor element is shown in accordance with various embodiments herein. A plurality of graphene varactors 302 can be disposed on the first measurement zone 204 in an array within the measurement zones. In some embodiments, a chemical sensor element can include a plurality of graphene varactors configured in an array. In some embodiments, the plurality of graphene varactors can include identical surface chemistries, while in other embodiments the plurality of graphene varactors can include different surface chemistries from one another. In some embodiments, graphene varactors having the same surface chemistries can be present in duplicate, triplicate, or more, such that data obtained during measurement cycles can be averaged together to further refine the change observed in the response signals. The graphene varactors herein can be as described in more detail in U.S. Pat. No. 9,513,244, which is herein incorporated by reference in its entirety. It will be appreciated that any of the first measurement zone, the second measurement zone, the third measurement zone, and the like can include one or more arrays of a plurality of graphene varactors as described herein.

In some embodiments, the graphene varactors can be heterogeneous in that they are different (in groups or as individual graphene varactors) from one another in terms of their binding behavior or specificity with regard a particular analyte. In some embodiments, some graphene varactors can be duplicated, triplicated, or more, for validation purposes but are otherwise heterogeneous from other graphene varactors. Yet in other embodiments, the graphene varactors can be homogeneous. While the graphene varactors 302 of FIG. 3 are shown as boxes organized into a grid, it will be appreciated that the graphene varactors can take on many different shapes (including, but not limited to, various polygons, circles, ovals, irregular shapes, and the like) and, in turn, the groups of graphene varactors can be arranged into many different patterns (including, but not limited to, star patterns, zig-zag patterns, radial patterns, symbolic patterns, and the like).

In some embodiments, the order of specific graphene varactors 302 across a length 312 and width 314 of the measurement zone can be substantially random. In other embodiments, the order can be specific. For example, in some embodiments, a measurement zone can be ordered so that the specific graphene varactors 302 configured to bind to analytes having a lower molecular weight are located farther away from the incoming gas flow relative to specific graphene varactors 302 configured to bind to analytes having a higher molecular weight which are located closer to the incoming gas flow. As such, chromatographic effects which may serve to provide separation between chemical compounds of different molecular weight can be taken advantage of to provide for optimal binding of chemical compounds to corresponding graphene varactors.

The number of graphene varactors can be from about 1 to about 100,000. In some embodiments, the number of graphene varactors can be from about 1 to about 10,000. In some embodiments, the number of graphene varactors can be from about 1 to about 1,000. In some embodiments, the number of graphene varactors can be from about 2 to about 500. In some embodiments, the number of graphene varactors can be from about 10 to about 500. In some embodiments, the number of graphene varactors can be from about 50 to about 500. In some embodiments, the number of graphene varactors can be from about 1 to about 250. In some embodiments, the number of graphene varactors can be from about 1 to about 50.

The graphene varactor or other graphene sensor herein can include a graphene layer and a self-assembled monolayer disposed on an outer surface of the graphene layer through π-π stacking interactions. Graphene is a form of carbon containing a single layer of carbon atoms in a hexagonal lattice. Graphene has a high strength and stability due to its tightly packed sp2 hybridized orbitals, where each carbon atom forms one sigma (σ) bond each with its three neighboring carbon atoms and has one p orbital projected out of the hexagonal plane. The p orbitals of the hexagonal lattice can hybridize to form a π bond suitable for non-covalent π-π stacking interactions with other π-electron rich molecules.

The non-covalent functionalization of graphene with a self-assembled monolayer does not significantly affect the atomic structure of graphene, and provides a stable graphene-based sensor with high sensitivity towards a number of volatile organic compounds (VOCs) in the parts-per-billion (ppb) or parts-per-million (ppm) levels. The use of specific compounds with which to form a self-assembled monolayer on the graphene can increase the sensitivity towards cyanide or other toxic compounds allowing for collection of a richer dataset to more accurately determine their presence.

As such, the graphene varactors described herein can include those in which a single graphene layer has been surface-modified through non-covalent π-π stacking interactions between graphene and π-electron-rich molecules.

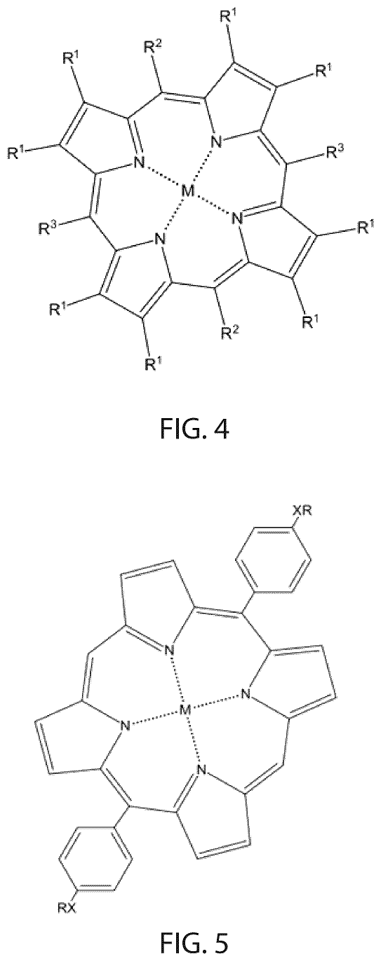

In some embodiments, the graphene varactors described herein can include those in which a single graphene layer has been surface-modified through non-covalent electrostatic interactions between the graphene layer and substituted metalloporphyrins. In some embodiments, the substituted metalloporphyrins herein can include those having the general formula shown in FIG. 4, where each R¹ is independently —H; —X, where X is a halogen atom; any linear or branched C₁-C₅₀ alkyl, C₁-C₅₀ alkenyl, C₁-C₅₀ alkynyl, C₁-C₅₀ heteroalkyl, C₁-C₅₀ heteroalkenyl, C₁-C₅₀ heteroalkynyl, or any combination thereof; —RZR, —ZRZR, or —RZRZR, where R can include, but not be limited to, any identical or different, linear or branched, C₁-C₅₀ alkyl, C₁-C₅₀ alkenyl, C₁-C₅₀ alkynyl, or any combination thereof, and Z can be one or more heteroatom selected from N, O, P, S, Se, or Si; —ROH, —RC(O)OH, —RC(O)OR, —ROR, —RSR, —RCHO, —RX, —RC(O)NH₂, —RC(O)NR, —RNH₃⁺, —RNH₂, —RNO₂, —RNR, —RNRR, —RB(OH)₂, or any combination thereof, where R can include, but is not to be limited to, any identical or different, linear, branched, or cyclic C₁-C₅₀ alkyl, C₁-C₅₀ alkenyl, C₁-C₅₀ alkynyl, or a combination thereof, or can be absent such that the remaining portion of the functional group is covalently bound directly to one or more carbon atoms of the substituted metalloporphyrin; and
where R² and R³ are independently —H; any linear or branched C₁-C₅₀ alkyl, C₁-C₅₀ alkenyl, C₁-C₅₀ alkynyl, C₁-C₅₀ heteroalkyl, C₁-C₅₀ heteroalkenyl, C₁-C₅₀ heteroalkynyl, or any combination thereof; —RZR, —ZRZR, or —RZRZR, where R can include, but not be limited to, any identical or different, linear or branched, C₁-C₅₀ alkyl, C₁-C₅₀ alkenyl, C₁-C₅₀ alkynyl, or any combination thereof, and Z can be one or more heteroatom selected from N, O, P, S, Se, or Si; an aryl, heteroaryl, substituted aryl, or substituted heteroaryl; a biphenyl or substituted biphenyl; an aryloxy, arylthio, arylamine, or any substitutions thereof; or any combination thereof; and
where every R^{1,} R^{2,} and R³ group is covalently bound directly to the substituted metalloporphyrin; and
where M is a metal including but not limited to aluminum, calcium, magnesium, manganese, iron, cobalt, nickel, zinc, ruthenium, palladium, silver, platinum, indium, tin, copper, rhodium, chromium, gallium, osmium, iridium, or derivatives thereof; and where the oxidation state of the metal can include, but not be limited to an oxidation state of I, II, III, IV, V, VI, VII, or VIII. In some embodiments, the metal is magnesium. In some embodiments the metal is silver. In some embodiments the metal is platinum. In some embodiments the metal is palladium. In some embodiments the metal is zinc. In some embodiments, various inorganic, organic, ionic, or neutral ligands can be coordinated with the metals herein, including, but not to be limited to, CO, NH₃, H₂O, NO, CH₃NH₂, pyridine, and the like. In some embodiments, the number of ligands can be from zero ligands to eight ligands.

In some embodiments, the R² and R³ groups are the same, while in other embodiments the R² and R³ groups are different.

In some embodiments, the substituted metalloporphyrins can include those having the general formula shown in FIG. 5, where each X is independently a heteroatom selected from N, O, P, S, Se, or Si, or absent, such that R is covalently attached to the phenyl group; and
where each R is independently —H; any linear or branched C₁-C₅₀ alkyl, C₁-C₅₀ alkenyl, C₁-C₅₀ alkynyl, C₁-C₅₀ heteroalkyl, C₁-C₅₀ heteroalkenyl, C₁-C₅₀ heteroalkynyl, or any combination thereof; —RZR, —ZRZR, or —RZRZR, where R can include, but not be limited to, any identical or different, linear or branched, C₁-C₅₀ alkyl, C₁-C₅₀ alkenyl, C1-C₅₀ alkynyl, or any combination thereof, and Z can be one or more heteroatom selected from N, O, P, S, Se, or Si; an aryl, heteroaryl, substituted aryl, or substituted heteroaryl;

a biphenyl or substituted biphenyl; an aryloxy, arylthio, arylamine, or any substitutions thereof; or any combination thereof; and
where M is a metal including but not limited to aluminum, calcium, magnesium, manganese, iron, cobalt, nickel, zinc, ruthenium, palladium, silver, platinum, indium, tin, copper, rhodium, chromium, gallium, osmium, iridium, or derivatives thereof; and where the oxidation state of the metal can include, but not be limited to an oxidation state of I, II, III, IV, V, VI, VII, or VIII. In some embodiments, the metal is magnesium. In some embodiments the metal is silver. In some embodiments the metal is platinum. In some embodiments the metal is palladium. In some embodiments the metal is zinc. In some embodiments, various inorganic, organic, ionic, or neutral ligands can be coordinated with the metals herein, including, but not to be limited to, Cl⁻, F⁻, Br⁻, I⁻, CN⁻, SCN⁻, CO, NH₃, H₂O, NO, CH₃NH₂, pyridine, and the like. In some embodiments, the number of ligands can be from zero ligands to eight ligands.

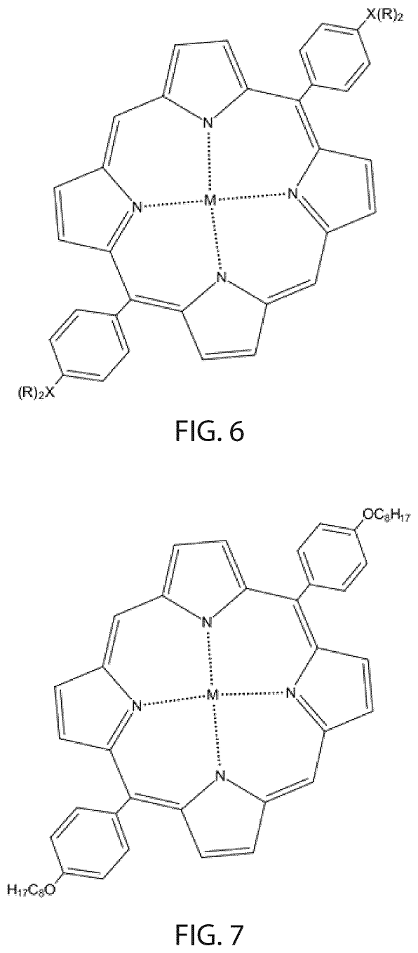

In some embodiments, the substituted metalloporphyrins can include those having the general formula shown in FIG. 6 where each X is independently a heteroatom selected from N, O, P, S, Se, or Si; and
where each R is independently —H; any linear or branched C₁-C₅₀ alkyl, C₁-C₅₀ alkenyl, C₁-C₅₀ alkynyl, C₁-C₅₀ heteroalkyl, C₁-C₅₀ heteroalkenyl, C₁-C₅₀ heteroalkynyl, or any combination thereof; —RZR, —ZRZR, or —RZRZR, where R can include, but not be limited to, any identical or different, linear or branched, C₁-C₅₀ alkyl, C₁-C₅₀ alkenyl, C₁-C₅₀alkynyl, or any combination thereof, and Z can be one or more heteroatom selected from N, O, P, S, Se, or Si; an aryl, heteroaryl, substituted aryl, or substituted heteroaryl; a biphenyl or substituted biphenyl; an aryloxy, arylthio, arylamine, or any substitutions thereof; or one R group can be absent; or any combination thereof; and
where M is a metal including but not limited to aluminum, calcium, magnesium, manganese, iron, cobalt, nickel, zinc, ruthenium, palladium, silver, platinum, indium, tin, copper, rhodium, chromium, gallium, osmium, iridium, or derivatives thereof; and where the oxidation state of the metal can include, but not be limited to an oxidation state of I, II, III, IV, V, VI, VII, or VIII. In some embodiments, the metal is magnesium. In some embodiments the metal is silver. In some embodiments the metal is platinum. In some embodiments the metal is palladium. In some embodiments the metal is zinc. In some embodiments, various inorganic, organic, ionic, or neutral ligands can be coordinated with the metals herein, including, but not to be limited to, Cl⁻, F⁻, Br⁻, I⁻, CN⁻, SCN⁻, CO, NH₃, H₂O, NO, CH₃NH₂, pyridine, and the like. In some embodiments, the number of ligands can be from zero ligands to eight ligands.

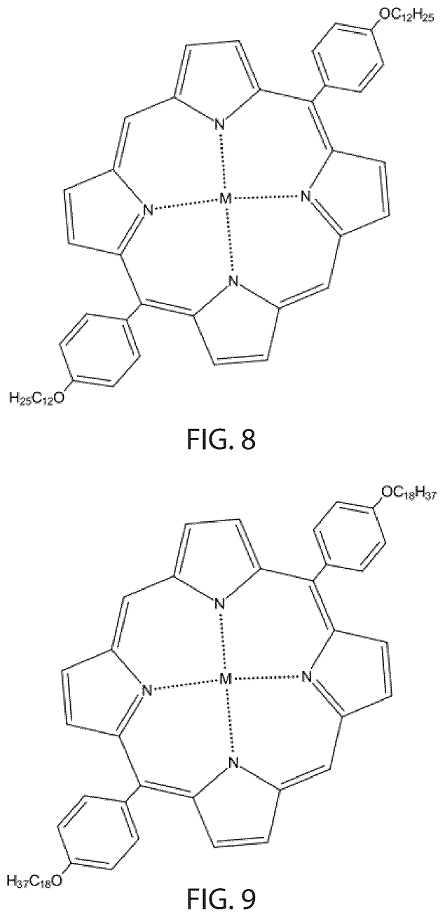

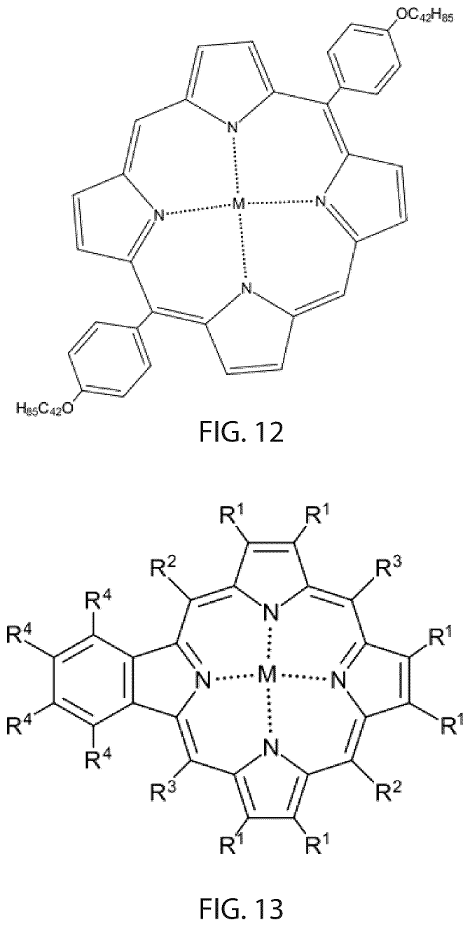

In some embodiments, suitable substituted metalloporphyrins can include those having formulas shown in FIGS. 7-12 where M is a metal including but not limited to aluminum, calcium, magnesium, manganese, iron, cobalt, nickel, zinc, ruthenium, palladium, silver, platinum, indium, tin, copper, rhodium, chromium, gallium, osmium, iridium, or derivatives thereof; and where the oxidation state of the metal can include, but not be limited to an oxidation state of I, II, III, IV, V, VI, VII, or VIII. In some embodiments, the metal is magnesium. In some embodiments the metal is silver. In some embodiments the metal is platinum. In some embodiments the metal is palladium. In some embodiments the metal is zinc. In some embodiments, various inorganic, organic, ionic, or neutral ligands can be coordinated with the metals herein, including, but not to be limited to, Cl⁻, F⁻, Br⁻, I⁻, CN⁻, SCN⁻, CO, NH₃, H₂O, NO, CH₃NH₂, pyridine, and the like. In some embodiments, the number of ligands can be from zero ligands to eight ligands.

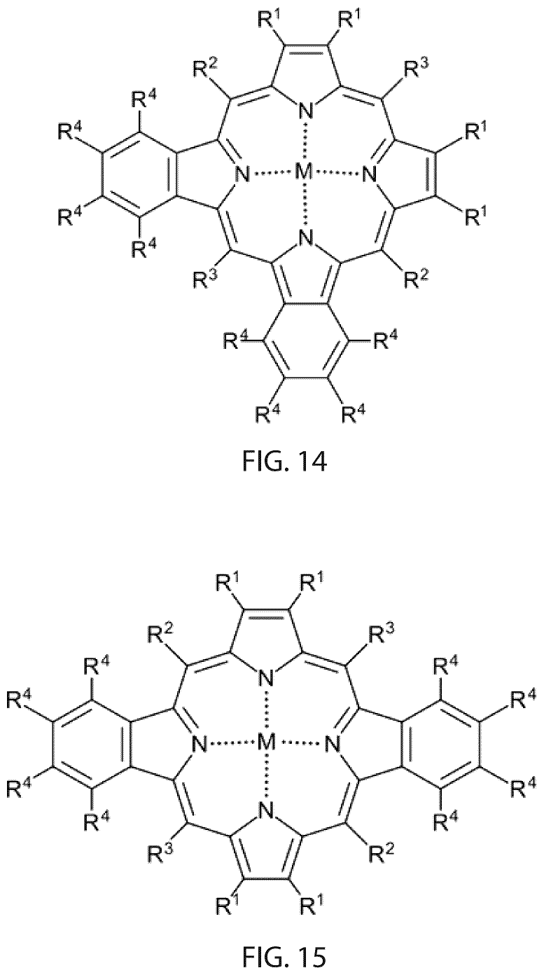

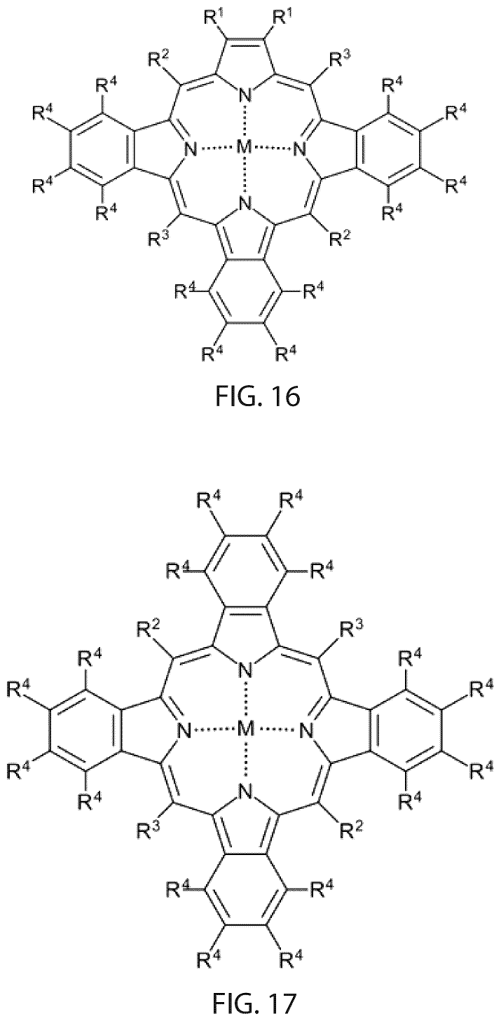

In some embodiments, the substituted metalloporphyrins can include those including phthalocyanine substitutions in the place of one or more of the pyrrole subunits of the metalloporphyrin ring structure. The phthalocyanine-substituted metalloporphyrins include those having the general formulas shown in FIGS. 13-17, where each R¹ and R⁴ are independently —H; —X, where X is a halogen atom; any linear or branched C₁-C₅₀ alkyl, C₁-C₅₀ alkenyl, C₁-C₅₀ alkynyl, C₁-C₅₀ heteroalkyl, C₁-C₅₀ heteroalkenyl, C₁-C₅₀ heteroalkynyl, or any combination thereof; —RZR, —ZRZR, or —RZRZR, where R can include, but not be limited to, any identical or different, linear or branched, C₁-C₅₀ alkyl, C₁-C₅₀ alkenyl, C₁-C₅₀ alkynyl, or any combination thereof, and Z can be one or more heteroatom selected from N, O, P, S, Se, or Si; —ROH, —RC(O)OH, —RC(O)OR, —ROR, —RSR, —RCHO, —RX, —RC(O)NH₂, —RC(O)NR, —RNH₃₊, —RNH₂, —RNO₂, —RNR, —RNRR, —RB(OH)₂, or any combination thereof, where R can include, but is not to be limited to, any identical or different, linear, branched, or cyclic C₁-C₅₀ alkyl, C₁-C₅₀ alkenyl, C₁-C₅₀ alkynyl, or a combination thereof, or can be absent such that the remaining portion of the functional group is covalently bound directly to one or more carbon atoms of the phthalocyanine-substituted metalloporphyrin; and
where R² and R³ are independently —H; any linear or branched C₁-C₅₀ alkyl, C₁-C₅₀ alkenyl, C₁-C₅₀ alkynyl, C₁-C₅₀ heteroalkyl, C₁-C₅₀ heteroalkenyl, C₁-C₅₀ heteroalkynyl, or any combination thereof; —RZR, —ZRZR, or —RZRZR, where R can include, but not be limited to, any identical or different, linear or branched, C₁-C₅₀ alkyl, C₁-C₅₀ alkenyl, C₁-C₅₀ alkynyl, or any combination thereof, and Z can be one or more heteroatom selected from N, O, P, S, Se, or Si; an aryl, heteroaryl, substituted aryl, or substituted heteroaryl; a biphenyl or substituted biphenyl; an aryloxy, arylthio, arylamine, or any substitutions thereof; or any combination thereof; and
where every R¹, R², R³, and R⁴ group is covalently bound directly to the phthalocyanine-substituted metalloporphyrin; and
where M is a metal including but not limited to aluminum, calcium, magnesium, manganese, iron, cobalt, nickel, zinc, ruthenium, palladium, silver, platinum, indium, tin, copper, rhodium, chromium, gallium, osmium, iridium, or derivatives thereof; and where the oxidation state of the metal can include, but not be limited to an oxidation state of I, II, III, IV, V, VI, VII, or VIII. In some embodiments, the metal is magnesium. In some embodiments the metal is silver. In some embodiments the metal is platinum. In some embodiments the metal is palladium. In some embodiments the metal is zinc. In some embodiments, various inorganic, organic, ionic, or neutral ligands can be coordinated with the metals herein, including, but not to be limited to, Cl⁻, F⁻, Br⁻, I⁻, CN⁻, SCN⁻, CO, NH₃, H₂O, NO, CH₃NH₂, pyridine, and the like. In some embodiments, the number of ligands can be from zero ligands to eight ligands.

In some embodiments, the R² and R³ groups are the same, while in other embodiments the R² and R³ groups are different.

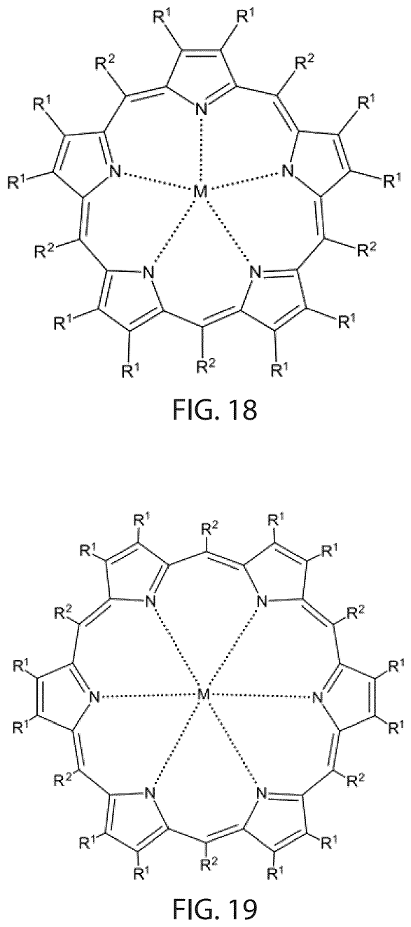

In some embodiments, the substituted metalloporphyrins herein can include one or more expanded substituted metalloporphyrins. As used herein, the term “expanded substituted metalloporphyrin” refers to a metalloporphyrin having an expanded ring structure including more than the four pyrrole rings associated with the structure shown in formula (11). In some embodiments, the expanded substituted metalloporphyrin can include 5 modified pyrrole subunits, each connected by a methine (═CH—) bridge. In some embodiments, the expanded substituted metalloporphyrin ring structure can include 6 modified pyrrole subunits, each connected by a methine (═CH—) bridge. In some embodiments, the expanded substituted metalloporphyrin ring structure can include more than 6 modified pyrrole subunits, each connected by a methine (═CH—) bridge. Exemplary expanded substituted metalloporphyrin molecules include, but are not to be limited to, those expanded substituted metalloporphyrin molecules having the general formulas as shown in FIGS. 18-19 where each R¹ is independently —H; —X, where X is a halogen atom; any linear or branched C₁-C₅₀ alkyl, C₁-C₅₀ alkenyl, C₁-C₅₀ alkynyl, C₁-C₅₀ heteroalkyl, C₁-C₅₀ heteroalkenyl, C₁-C₅₀ heteroalkynyl, or any combination thereof; —RZR, —ZRZR, or —RZRZR, where R can include, but not be limited to, any identical or different, linear or branched, C₁-C₅₀ alkyl, C₁-C₅₀ alkenyl, C₁-C₅₀ alkynyl, or any combination thereof, and Z can be one or more heteroatom selected from N, O, P, S, Se, or Si; —ROH, —RC(O)OH, —RC(O)OR, —ROR, —RSR, —RCHO, —RX, —RC(O)NH₂, —RC(O)NR, —RNH₃₊, —RNH₂, —RNO2, —RNR, —RNRR, —RB(OH)₂, or any combination thereof, where R can include, but is not to be limited to, any identical or different, linear, branched, or cyclic C₁-C₅₀ alkyl, C₁-C₅₀ alkenyl, C₁-C₅₀ alkynyl, or a combination thereof, or can be absent such that the remaining portion of the functional group is covalently bound directly to one or more carbon atoms of the expanded substituted metalloporphyrin; and
where R² is independently —H; any linear or branched C₁-C₅₀ alkyl, C₁-C₅₀ alkenyl, C₁-C₅₀ alkynyl, C₁-C₅₀ heteroalkyl, C₁-C₅₀ heteroalkenyl, C₁-C₅₀ heteroalkynyl or any combination thereof; —RZR, —ZRZR, or —RZRZR, where R can include, but not be limited to, any identical or different, linear or branched, C₁-C₅₀ alkyl, C₁-C₅₀ alkenyl, C₁-C₅₀ alkynyl, or any combination thereof, and Z can be one or more heteroatom selected from N, O, P, S, Se, or Si; an aryl, heteroaryl, substituted aryl, or substituted heteroaryl; a biphenyl or substituted biphenyl; an aryloxy, arylthio, arylamine, or any substitutions thereof; or any combination thereof; and
where every R¹ and R² group is covalently bound directly to the expanded substituted metalloporphyrin; and
where M is a metal including but not limited to aluminum, calcium, magnesium, manganese, iron, cobalt, nickel, zinc, ruthenium, palladium, silver, platinum, indium, tin, copper, rhodium, chromium, gallium, osmium, iridium, or derivatives thereof; and where the oxidation state of the metal can include, but not be limited to an oxidation state of I, II, III, IV, V, VI, VII, or VIII. In some embodiments, the metal is magnesium. In some embodiments the metal is silver. In some embodiments the metal is platinum. In some embodiments the metal is palladium. In some embodiments the metal is zinc. In some embodiments, various inorganic, organic, ionic, or neutral ligands can be coordinated with the metals herein, including, but not to be limited to, Cl⁻, F⁻, Br⁻, I⁻, CN⁻, SCN⁻, CO, NH₃, H₂O, NO, CH₃NH₂, pyridine, and the like. In some embodiments, the number of ligands can be from zero ligands to eight ligands.

It will be appreciated that the expanded substituted metalloporphyrin ring structures described herein can further include those having phthalocyanine substitutions in the place of one or more of the pyrrole subunits of the porphyrin ring structures of the formulas shown in FIGS. 18 and 19.

Other compounds that can be used to modify graphene surfaces herein can include, for example, at least one of pyrenes, coronenes, aromatic cyclodextrins, and pillarenes. In some embodiments, at least two of pyrenes, coronenes, aromatic cyclodextrins, and pillarenes are included on graphene surfaces herein. In some embodiments, at least three of pyrenes, coronenes, aromatic cyclodextrins, and pillarenes are included on graphene surfaces herein. In some embodiments, at least four of pyrenes, coronenes, aromatic cyclodextrins, and pillarenes are included on graphene surfaces herein. Aromatic cyclodextrins can include benzylated cyclodextrins, such as α-, β- and γ-cyclodextrins derivatives, including, but not limited to, α-CDBn18, β-CDBn21, γ-CDBn24, β-CDBn19(OH)2. In some embodiments, pyrenes herein can include acids, such as pyrene-1-boronic acid as well as other acids.

In some embodiments, the self-assembled monolayer can provide at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% surface coverage (by area) of the graphene layer. It will be appreciated that the self-assembled monolayer can provide surface coverage falling within a range wherein any of the forgoing percentages can serve as the lower or upper bound of the range, provided that the lower bound of the range is a value less than the upper bound of the range.

In some embodiments, it will be appreciated that the self-assembly of π-electron-rich molecules on the surface of the graphene layer can include the self-assembly into more than a monolayer, such as a multilayer. Multilayers can be detected and quantified by techniques such as scanning tunneling microscopy (STM) and other scanning probe microscopies. References herein to a percentage of coverage greater than 100% shall refer to the circumstance where a portion of the surface area is covered by more than a monolayer, such as covered by two, three or potentially more layers of the compound used. Thus, a reference to 105% coverage herein shall indicate that approximately 5% of the surface area includes more than monolayer coverage over the graphene layer. In some embodiments, graphene surfaces can include 101%, 102%, 103%, 104%, 105%, 110%, 120%, 130%, 140%, 150%, or 175% surface coverage of the graphene layer. It will be appreciated that multilayer surface coverage of the graphene layer can fall within a range of surface coverages, wherein any of the forgoing percentages can serve as the lower or upper bound of the range, provided that the lower bound of the range is a value less than the upper bound of the range. For example, ranges of coverage can include, but are not limited to, 50% to 150% by surface area, 80% to 120% by surface area, 90% to 110%, or 99% to 120% by surface area.

In some embodiments, the self-assembled monolayers suitable for use herein can provide coverage of the graphene surface with a monolayer as quantified by the Langmuir theta value of at least some minimum threshold value, but avoid covering the majority of the surface of the graphene with a multilayer thicker than a monolayer. In some embodiments, the self-assembled monolayers suitable for use herein provide a Langmuir theta value of at least 0.95. In some embodiments, the self-assembled monolayers suitable for use herein provide a Langmuir theta value of at least 0.98. In some embodiments, the self-assembled monolayers can provide a Langmuir theta value of at least 0.85, 0.86, 0.87, 0.88, 0.89, 0.90, 0.91, 0.92, 0.93, 0.94, 0.95, 0.96, 0.97, 0.98, 0.99, or 1.0. It will be appreciated that the self-assembled monolayer can provide a range of Langmuir theta values, wherein any of the forgoing Langmuir theta values can serve as the lower or upper bound of the range, provided that the lower bound of the range is a value less than the upper bound of the range.

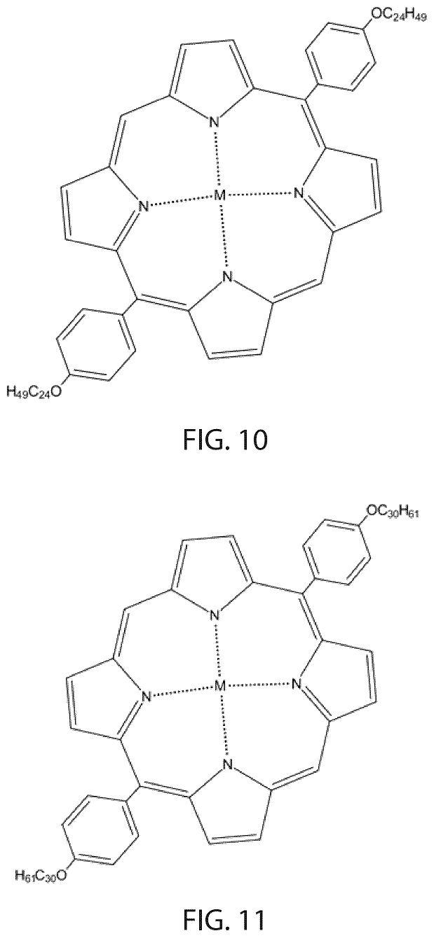

In some embodiments, each of the graphene varactors suitable for use herein can include at least a portion of one or more electrical circuits. By way of example, in some embodiments, each of the graphene varactors can include all or a portion of one or more passive electrical circuits or active electrical circuits. In some embodiments, the graphene varactors herein can include two-terminal graphene varactors. In some embodiments, the two-terminal graphene varactors can be adapted to each receive independent signals from an electrical signal generator. In some embodiments, the graphene varactors can be formed such that they are integrated directly on an electronic circuit. In some embodiments, the graphene varactors can be formed such that they are wafer bonded to the circuit. In some embodiments, the graphene varactors can include integrated readout electronics, such as a readout integrated circuit (ROIC). The electrical properties of the electrical circuit, including resistance or capacitance, can change upon binding, such as specific and/or non-specific binding, with a compound from a biological sample. Many different types of circuits can be used to gather data from chemical sensor elements and will be discussed below in reference to FIGS. 9 and 10.

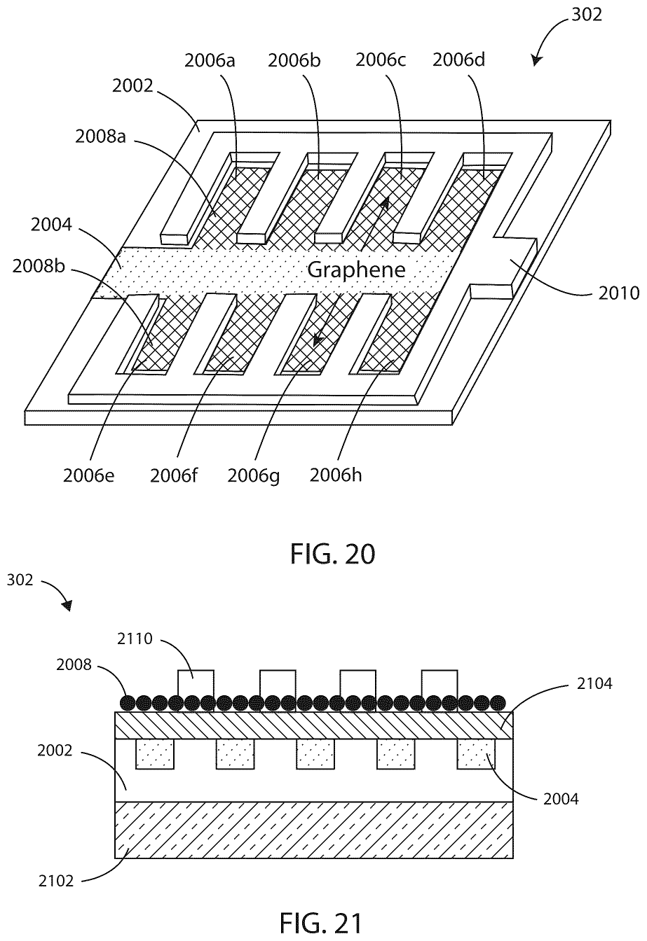

Referring now to FIG. 20, a schematic view of a graphene varactor 302 having two terminals is shown in accordance with the embodiments herein. It will be appreciated that graphene varactors can be prepared in various ways with various geometries, and that the graphene varactor shown in FIG. 20 is just one example in accordance with the embodiments herein.

Graphene varactor 302 can include an insulator layer 2002, a gate electrode 2004 (or “gate contact”), a dielectric layer (item 2104 in FIG. 21), one or more graphene layers, such as graphene layers 2008a and 2008b, and a contact electrode 2010 (or “graphene contact”). In some embodiments, the graphene layer(s) 2008a -b can be contiguous, while in other embodiments the graphene layer(s) 2008a -b can be non-contiguous. Gate electrode 2004 can be deposited within one or more depressions formed in insulator layer 2002. Insulator layer 2002 can be formed from an insulative material such as silicon dioxide, formed on a substrate (item 2102 in FIG. 21), such as a silicon substrate (wafer), and the like. Gate electrode 2004 can be formed by an electrically conductive material such as chromium, copper, gold, silver, tungsten, aluminum, titanium, palladium, platinum, iridium, and any combinations or alloys thereof, which can be deposited on top of or embedded within the insulator layer 2002. The dielectric layer (not shown in FIG. 20) can be disposed on a surface of the insulator layer 2002 and the gate electrode 2004, as shown in more detail in FIG. 21. The graphene layer(s) 2008a -b can be disposed on the dielectric layer.

Graphene varactor 302 includes eight gate electrode fingers 2006a -2006h. It will be appreciated that while graphene varactor 302 shows eight gate electrode fingers 2006a -2006h, any number of gate electrode finger configurations can be contemplated. In some embodiments, an individual graphene varactor can include fewer than eight gate electrode fingers. In some embodiments, an individual graphene varactor can include more than eight gate electrode fingers. In other embodiments, an individual graphene varactor can include two gate electrode fingers. In some embodiments, an individual graphene varactor can include 1, 2, 3, 4, 5, 6, 7, 8, 9, 10, or more gate electrode fingers.

Graphene varactor 302 can include one or more contact electrodes 2010 disposed on portions of the graphene layers 2008a and 2008b. Contact electrode 2010 can be formed from an electrically conductive material such as chromium, copper, gold, silver, tungsten, aluminum, titanium, palladium, platinum, iridium, and any combinations or alloys thereof. Further aspects of exemplary graphene varactors can be found in U.S. Pat. No. 9,513,244, the content of which is herein incorporated by reference in its entirety.

Referring now to FIG. 21, a schematic diagram of a portion of a cross-sectional view of an exemplary graphene varactor is shown in accordance with various embodiments herein. The graphene varactor can include a substrate 2102, such as a silicon substrate (wafer). An insulator layer 2002 can be disposed on the substrate 2102, and a gate electrode 2004 can be recessed into the insulator layer 2002. The gate electrode 2004 can be formed by depositing an electrically conductive material in the depression in the insulator layer 2002, as discussed above in reference to FIG. 20. A dielectric layer 2104 can be formed on a surface of the insulator layer 2002 and the gate electrode 2004. In some examples, the dielectric layer 2104 can be formed of a material, such as, silicon dioxide, aluminum oxide, hafnium dioxide, zirconium dioxide, hafnium silicate, or zirconium silicate. The graphene layer 2008 is disposed on the dielectric layer 2104 and the contact electrode 2010 can be disposed in contact with the graphene layer 2008. In some examples, the dielectric layer 2104 can include multiple layers of the dielectric materials listed herein. In some embodiments, the dielectric layer 2104 can include alternating layers of different dielectric materials. In some embodiments, the dielectric layer 2104 can include alternating layers of aluminum oxide and hafnium dioxide.

In some embodiments herein, to maintain the stability of the graphene varactors herein, the chemical sensor elements can be pretreated under a vacuum at a temperature from 50° C. to 150° C. for at least 3 hours. In various embodiments, the chemical sensor elements can be pretreated under vacuum at a temperature from 120° C. to 150° C. for 10 to 20 hours. In various embodiments, the chemical sensor elements can be pretreated under a vacuum at a temperature can be greater than or equal to 50° C., 60° C., 70° C., 80° C., 90° C., 100° C., 110° C., 120° C., 130° C., 140° C., 150° C., 160° C., 170° C., 180° C., 190° C., or 200°° C., or can be an amount falling within a range between any of the foregoing.

In addition, the chemical sensor elements herein can be maintained under a controlled gas environment until it is exposed to a gaseous test sample. By way of example, the chemical sensor element can be maintained under a controlled gas environment including oxygen gas, nitrogen gas or an inert gas such as, for example, argon, helium, xenon, krypton, or neon.

In many cases, each graphene varactor can be interrogated using a number of different applied voltages (a plurality of voltages) with the resulting data forming a C-Vg curve. The plurality of voltages can fall within a range from a lower voltage bound to an upper voltage bound. In many cases the voltages may start at the lower bound and then be increased progressing up to the upper bound, thus sweeping across the range in a first direction followed by a sweep in the opposite (or second) direction (e.g., from the upper bound to the lower bound). Thus, in various embodiments, a first direction can include a sweep from the lower voltage bound to the upper voltage bound and a second direction is a sweep from the upper voltage bound to the lower voltage bound. However, in other embodiments, the first direction can include a sweep from the upper voltage bound to the lower voltage bound and the second direction is a sweep from the lower voltage bound to the upper voltage bound. In various embodiments, a sweep in the first direction followed by a sweep in the second direction constitutes a measurement cycle.

The values for the lower voltage bound and the upper voltage bounds can be predetermined or can be determined dynamically. In various embodiments, the lower voltage bound and the upper voltage bound are preset values and can be selected from values such as −3.0 V or less, −2.9 V, −2.8 V, −2.7 V, −2.6 V, −2.5 V, −2.4 V, −2.3 V, −2.2 V, −2.1 V, −2.0 V, −1.9 V, −1.8 V, −1.7 V, −1.6 V, −1.5 V, −1.4V, −1.3 V, −1.2V, −1.1 V, −1.0 V, −0.9 V, −0.8 V, −0.7 V, −0.6 V, −0.5 V, −0.4V, −0.3 V, −0.2 V, −0.1 V, 0V, 0.1 V, 0.2 V, 0.3 V, 0.4 V, 0.5 V, 0.6 V, 0.7 V, 0.8 V, 0.9 V, 1.0V, 1.1 V, 1.2 V, 1.3 V, 1.4 V, 1.5 V, 1.6 V, 1.7 V, 1.8 V, 1.9 V, 2.0 V, 2.1 V, 2.2 V, 2.3 V, 2.4 V. 2.5 V, 2.6 V, 2.7 V, 2.8 V, 2.9 V, or 3.0 V or more, or a voltage value falling between any of the foregoing values. In various embodiments, the lower voltage bound and the upper voltage bound are preset values and can be selected from values ranging from −5 V to 5 V; from −4 V to 4 V; from −3 V to 3 V; from −2 V to 2 V; from −1.5 V to 1.5V; or from −1 V to 1 V.

While an instantaneous applied voltage herein can be thought of as the sum of a DC bias component and an AC component, it will be appreciated that specific applied voltages values as referenced herein typically represent the DC voltage bias or offset value. This is because the average value of an AC component over a non-instantaneous time period will be zero. As such, unless otherwise stated to the contrary or the context dictates otherwise, voltage value references herein shall refer to the DC bias or offset component of an applied voltage, understanding that corresponding instantaneous voltage values can vary based on the AC component. The waveforms of the AC component can take many different forms. For example, they can be sinusoidal, square, triangular, trapezoidal, ramped, sawtooth, complex, or the like.

In some embodiments, the lower voltage bound and the upper voltage bound are dynamically determined values. For example, the bounds can be changed based on previously applied excitation voltages and/or previously observed values related to the graphene sensor and/or previously observed effects.

In some embodiments the upper voltage bound and the lower voltage bound is static between successive measurement cycles. In other embodiments, the upper voltage bound and the lower voltage bound may change between successive measurement cycles. For example, in some embodiments, the first measurement cycle can include the use of the widest range of excitation voltages and successive measurement cycles may utilize a narrower range of excitation voltages.

Various timing schemes can be used for the sweep across a range of voltages. In some embodiments, a sweep in the first direction can be immediately followed by a sweep in the second direction. In other embodiments, a sweep in the first direction can be followed by a pause and then a sweep in the second direction. The duration of a pause between sweeps can include those from 1 millisecond (ms) to 5 seconds in length. In some embodiments, the duration of the pause between sweeps can be greater than or equal to, 10 ms, 20 ms, 30 ms, 40 ms, 50 ms, 60 ms, 70 ms, 80 ms, 90 ms, 100 ms, 200 ms, 300 ms, 400 ms, 500 ms, 600 ms, 700 ms, 800 ms, 900 ms, or 1 sec, 2 sec, 3 sec, 4 sec, or 5 sec, or can be an amount falling within a range between any of the foregoing.

In some embodiments, the duration of a pause between sweeps can be greater than 5 seconds in length. In various embodiments, the duration of a pause between sweeps can greater than or equal to 6 sec, 7 sec, 8 sec, 9 sec, 10 sec, 11 sec, 12 sec, 13 sec, 14 sec, 15 sec, 16 sec, 17 sec, 18 sec, 19 sec, 20 sec, 21 sec, 22 sec, 23 sec, 24 sec, 25 sec, 26 sec, 27 sec, 28 sec, 29 sec, 30 sec, 31 sec, 32 sec, 33 sec, 34 sec, 35 sec, 36 sec, 37 sec, 38 sec, 39 sec, 40 sec, 41 sec, 42 sec, 43 sec, 44 sec, 45 sec, 46 sec, 47 sec, 48 sec, 49 sec, 50 sec, 51 sec, 52 sec, 53 sec, 54 sec, 55 sec, 56 sec, 57 sec, 58 sec, 59 sec, or 60 sec, or can be an amount falling within a range between any of the foregoing. In other embodiments, the duration of a pause between sweeps can be greater than 1 minute.

A change in any one of the parameters of the capacitance versus voltage values provides data that can reflect the binding status of analytes to the graphene varactor(s), and can be used to characterize a sample and/or distinguish various analytes and analyte concentrations in the sample.

Various measurable aspects can be used to characterize the content of a sample (such as a breath sample) herein to determine the presence of cyanide or other toxic compounds and serve as data for use in estimating a time of ingestion of the same. In some embodiments, a ratio of the maximum capacitance to minimum capacitance can be used to characterize the content of a gaseous mixture. In some embodiments, a ratio of the maximum capacitance to the shift in the Dirac point can be used to characterize the content of a gaseous mixture. In other embodiments, a ratio of the minimum capacitance to the shift in the slope of the response signal can be used to characterize the content of a gaseous mixture. In some embodiments, a ratio of any of the parameters including a shift in the Dirac point, a change in the minimum capacitance, a change in the slope of the response signal, or the change in the maximum capacitance can be used to characterize the content of a sample mixture. In accordance with embodiments herein, hysteresis effects observed with respect to any of these values (as well as other types of values discussed) can be used to characterize the content of sample mixtures. In various embodiments, cyanide compounds and/or other toxic compounds can specifically result in a change in the minimum capacitance of the Dirac point (such as an increase in the minimum capacitance).

Various measurement circuitry can be used to measure the changes in the parameters of the capacitance-voltage curve of the graphene varactor(s). Measurement circuitry suitable for use herein can include active and passive sensing circuits. Such circuitry can implement wired (direct electrical contact) or wireless sensing techniques.

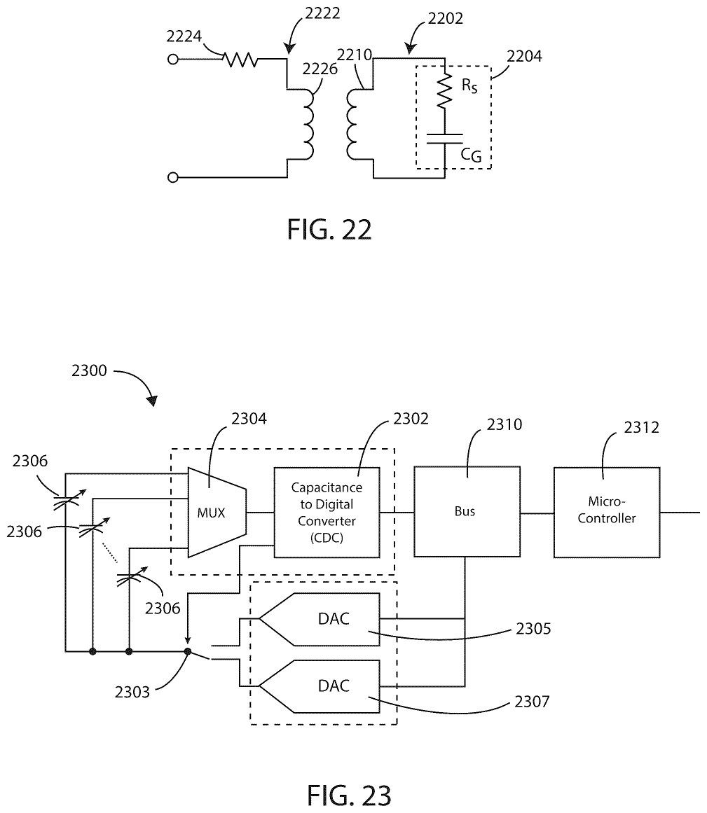

Referring now to FIG. 22, a schematic diagram of a passive sensor circuit 2202 and a portion of a reading circuit 2222 is shown in accordance with various aspects herein. In some embodiments, the passive sensor circuit 2202 can include a metal-oxide-graphene varactor 2204 (wherein RS represents the series resistance and CG represents the varactor capacitor) coupled to an inductor 2210. In some embodiments, the reading circuit 2222 can include a reading coil having a resistance 2224 and an inductance 2226.

Measurement circuitry herein can also include active sensing circuits. In various embodiments, the measurement circuity can include an electrical signal generator configured to generate a series of measurement cycles over a time period. The measurement circuity can include an electrical signal generator configured to generate and deliver an applied voltage that can be represented as an alternating voltage (or excitation voltage) superimposed on a bias voltage. It will be appreciated that there are many ways to generate such an applied voltage.

In some embodiments, measurement circuity can include an electrical signal generator configured to generate and deliver an applied voltage that includes a sinusoidal, square, triangular, trapezoidal, ramped, sawtooth, or complex waveform alternating voltage superimposed on a bias voltage. In some embodiments, the electrical signal generator can be configured to generate an applied voltage at a plurality of voltages to be applied to the one or more graphene varactors, the voltages falling within a range from a lower voltage bound and an upper voltage bound, the voltages starting at one bound and moving to the other bound as part of a sweep across the voltages. In some embodiments, the electrical signal generator can be configured to generate an excitation current at a plurality of voltages to be applied to the one or more graphene varactors, the voltages falling within a range from a lower bound and an upper bound, the voltages starting at one bound and moving to the other bound as part of a sweep across the voltages.

Referring now to FIG. 23, a schematic diagram is shown of an example of measurement circuity 2300 to measure the capacitance of a plurality of graphene sensors in accordance with various embodiments herein. The measurement circuity 2300 can include a capacitance to digital converter (CDC) 2302 in electrical communication with a multiplexor 2304. The multiplexor 2304 can provide selective electrical communication with a plurality of graphene varactors 2306. The connection to the other side of the graphene varactors 2306 can be controlled by a switch 2303 (as controlled by the CDC) and can provide selective electrical communication with a first digital to analog converter (DAC) 2305 and a second digital to analog converter (DAC) 2307. The other side of the DACs 2305, 2307 can be connected to a bus device 2310, or in some cases, the CDC 2302. The circuitry can further include a microcontroller 2312 (or controller circuit), which will be discussed in more detail below.

In this case, a signal from the CDC controls the switch 2303 between the output voltages of the two programmable Digital to Analog Converters (DACs) 2305 and 2307. The programmed voltage difference between the DACs determines an excitation amplitude (and represents the AC component of the applied voltage), providing an additional programmable scale factor to the measurement and allowing measurement of a wider range of capacitances than specified by the CDC. The bias voltage at which the capacitance is measured is equal to the difference between the bias voltage at the CDC input (via the multiplexor, usually equal to VCC/2, where VCC is the supply voltage) and the average voltage of the excitation signal, which is programmable. In some embodiments, buffer amplifiers and/or bypass capacitance can be used at the DAC outputs to maintain stable voltages during switching. It will be appreciated that the circuits of FIGS. 22 and 23 are merely exemplary. Many different approaches are contemplated herein.

The measurement circuity can include a capacitance sensor configured to measure capacitance of the discrete graphene components resulting from the excitation voltage.

The measurement circuity can also include a controller circuit configured to determine a change in at least one of a measured capacitance versus voltage value and a calculated value based on the measured capacitance or voltage over the time period. In various embodiments, the measured capacitance versus voltage values can include one or more of a capacitance at a particular voltage, a maximum slope of capacitance to voltage, a minimum slope of capacitance to voltage, a minimum capacitance, a voltage at minimum capacitance (Dirac voltage), a maximum capacitance, and a ratio of maximum capacitance to minimum capacitance. In various embodiments, the controller circuit is configured to measure a difference between a forward Dirac point voltage and a reverse Dirac point voltage. In some embodiments, the controller circuit is configured to calculate a rate of change of measured capacitance over the time period at multiple discrete DC bias voltages. In some embodiments, the controller circuit is configured to calculate an average hysteresis change value of a measured property over a plurality of measurement cycles. In various embodiments, the controller circuit is configured to determine the forward Dirac point voltage and/or the reverse Dirac point voltage.

In some embodiments, the measurement circuitry or another part of the system herein can include a temperature controller configured to control a temperature of the graphene varactors. In some embodiments, the temperature controller can include a thermistor, thermocouple, resistive thermal device (RTD) and the like. In various embodiments, controlling the temperature of the graphene varactors comprises exposing the graphene varactor to one or more temperature set points for a predetermined time. In some embodiments, a sequence involving increasing the temperature set points over a course of a predetermined time can be used. In other embodiments, a sequence involving decreasing the temperature set points over a course of a predetermined time can be used. In other embodiments, a sequence involving increasing the temperature set points followed by decreasing the temperature set points can be used.

The system for measuring analyte presence in a gaseous sample can be configured to measure differences in a capacitance versus voltage value when an applied voltage is swept in a first direction between the lower voltage bound and upper voltage bound versus a second direction between the between the upper voltage bound and lower voltage bound. In various embodiments, the first direction is a sweep from the lower voltage bound to the upper voltage bound and the second direction is a sweep from the upper voltage bound to the lower voltage bound. In various embodiments, the first direction is a sweep from the upper voltage bound to the lower voltage bound and the second direction is a sweep from the lower voltage bound to the upper voltage bound.

Various values for the voltages suitable for use within a range from a lower bound to an upper bound as contemplated herein are described further below. In various embodiments, each measurement cycle includes delivering a DC bias voltage to the discrete graphene components at multiple discrete DC bias voltage values across a range of DC bias voltages as discussed in greater detail below.

In some cases, the above calculated values can be indicative of the identity and/or concentrations of specific components of a gas sample, such as cyanide compounds. In some cases, each of the calculated values above can serve as a distinct piece of data that forms part of a pattern for a given subject and/or given gas sample. As also described elsewhere herein, the pattern can then be matched against preexisting patterns, or patterns identified in real-time, derived from large, stored data sets through techniques such as machine learning or other techniques, wherein such patterns are determined to be characteristic of toxic compounds such as cyanide compounds. The above calculated aspects can also be put to other purposes, diagnostic and otherwise. In some cases herein, patterns relevant for detecting the presence of cyanide include a pattern including a response in those sensor components that are sensitive to cyanide (such as those modified with metalloporphryins such as iron and cobalt metalloporphryins). In some cases herein, patterns relevant for detecting the presence of cyanide include a pattern including a response in those sensor components that are sensitive to cyanide (such as those modified with metalloporphryins such as iron and cobalt metalloporphryins) and a lesser response or a lack of a response (a differential response pattern) in those sensor components that are not sensitive to cyanide (such as those modified with non-polar compounds such as pyrene and/or coronene). When such pattern(s) are detected, the system can indicate that cyanide (or another toxic compound) is present and/or that cyanide or other toxic compound poisoning has occurred.

In some embodiments, calculations such as those described above can be performed by a controller circuit. The controller circuit can be configured to receive an electrical signal reflecting the capacitance or voltage of the graphene varactors. In some embodiments, the controller circuit can include a microcontroller to perform these calculations. In some embodiments, the controller circuit can include a microprocessor in electrical communication with the measurement circuity. The microprocessor system can include components such as an address bus, a data bus, a control bus, a clock, a CPU, a processing device, an address decoder, RAM, ROM and the like. In some embodiments, the controller circuit can include a calculation circuit (such as an application specific integrated circuit-ASIC) in electrical communication with the measurement circuity.

In addition, in some embodiments, the system can include a nonvolatile memory. In some embodiments, the non-volatile memory can be configured to store measured capacitance values for the discrete graphene components across a range of DC bias voltages. In other embodiments, the nonvolatile memory can be configured to store a baseline capacitance for the discrete graphene components across a range of DC bias voltages. In some embodiments, the nonvolatile memory can be where sensitivity calibration information for the graphene varactors is stored.

By way of example, the graphene varactors can be tested in a production facility, where sensitivity to various analytes such as VOC's can be determined and then stored on an EPROM or similar component. In addition, or alternatively, sensitivity calibration information can be stored in a central database and referenced with a chemical sensor element serial number when subject data is sent to a central location for analysis and diagnosis. These components can be included with any of the pieces of hardware described herein.

In some embodiments herein, components can be configured to communicate over a network, such as the internet or a similar network. In various embodiments, a central storage and data processing facility can be included. In some embodiments, data gathered from sensors in the presence of the subject (local) can be sent to the central processing facility (remote) via the internet or a similar network, and the pattern from the particular subject being evaluated can be compared against those reflecting the presence of toxic compounds such as cyanide.

### Pattern Matching

As referenced above, patterns relevant for detecting the presence of a poison (such as cyanide) can include a pattern including a response in those sensor components that are sensitive to the poison (such as, in the case of cyanide, those modified with metalloporphryins such as iron and cobalt metalloporphryins). Patterns relevant for detecting the presence of cyanide can also include a pattern including a response in those sensor components that are sensitive to cyanide (such as those modified with metalloporphryins such as iron and cobalt metalloporphryins) and a lesser response or a lack of a response in those sensor components that are not sensitive to cyanide (such as those modified with non-polar compounds such as pyrene and/or coronene).

Similarly, patterns relevant for detecting the presence of an overdosed compound and/or combinations of compounds (such as fentanyl and/or combinations of fentanyl with other compounds) can include a pattern including a response in those sensor components that are sensitive to the overdosed compound. Patterns relevant for detecting the presence of an overdosed compound can also include a pattern including a response in those sensor components that are sensitive to the overdosed compound and a lesser response or a lack of a response in those sensor components that are not sensitive to the overdosed compound.

In some embodiments, pattern matching algorithms can be used to match the current subject's pattern against predetermined patterns that correlate with (and can therefore indicate) the presence of, estimated amounts of, and/or time of exposure to compounds. Thus, in various embodiments herein, the system can compare a data set reflecting a particular patient/individual against one or more previously determined patterns using a pattern matching or pattern recognition algorithm to determine the pattern that is the best match, wherein the specific previously determined pattern that is the best match indicates one or more of the presence of, estimated amounts of, and/or time of exposure to compounds such as poisons (such as cyanide), overdosed compounds (such as fentanyl), and/or combinations of compounds.

Algorithms can be used herein to create new patterns/models using any of numerous machine learning techniques, or apply the results of previously calculated models using these techniques, such as logistic regression, random forest, or an artificial neural network. Many different pattern matching or pattern recognition algorithms can be used. By way of example, in some embodiments a least squares algorithm can be used to identify a particular pre-determined pattern that a combined data set most closely matches. In various embodiments, standard pattern classification methods can be used including, but not limited to, Gaussian mixture models, clustering, hidden Markov models, as well as Bayesian approaches, neural network models, and deep learning.

After a pattern matching operation herein, one or more of the presence of, estimated amounts of, and/or time of exposure to a toxic compound (such as a cyanide compound) can be determined. In some embodiments this can be performed remotely and provided back across the data network to the facility where the subject is currently located. In other embodiments, such operations can be performed locally on a device of the system.

### Applied Voltages/Measurement Cycles

A measurement cycle herein can include a sweep through voltages in a first direction followed by a sweep through voltages in the opposite direction to observe measurable parameters and/or changes in measurable parameters. Many different ranges of applied voltages can be used for each measurement cycle. In some embodiments, the applied voltages used in the methods herein can include from −6.0 V, −5.0 V, −4.0 V, −3.0V, −2.5 V, −2.0 V, −1.5 V, −1.0 V, −0.5 V, 0.5 V, 1.0 V, 1.5 V, 2.0 V, 2.5 V, 3.0 V, 4.0 V, 5.0 V, and 6.0 V. It will be appreciated that the applied voltages used in the methods herein can include delivering an applied voltage within a range, wherein any of the forgoing voltages can serve as the lower or upper bound of the range, provided that the lower bound of the range is a value less than the upper bound of the range.

In various embodiments, a “sweep” across a voltage range can include a number of discrete measurements being made during the sweep at a number of discrete bias voltages across the voltage range. In some embodiments, a measurement cycle herein can include a forward sweep (from low applied voltages to high applied voltages). In some embodiments, a measurement cycle herein can include a backward sweep (from high applied voltages to low applied voltages). In some embodiments, a measurement cycle herein can include both a forward and backward sweep. In some embodiments, a measurement cycle herein can include both a forward and backward sweep or any combination thereof.

In some embodiments, a voltage of 0 V or 0.5 V (or other “reset” voltage) can be applied at the end of a measurement cycle and before the next measurement cycle or at the end of all testing.

The length of time for each measurement cycle can depend on various factors including the total number of measurements made of capacitance during the cycle, the total bias voltage range being covered, the voltage step size for each measurement, the time for each measurement, etc. In some embodiments, the time period for each measurement cycle can be about 0.1, 0.5, 1, 2, 3, 4, 5, 6, 7, 8, 9, 10, 12, 15, 20, 30, 45, 60, 120 seconds or more. It will be appreciated that the time period for each measurement cycle can include a range, wherein any of the forgoing time points can serve as the lower or upper bound of the range, provided that the lower bound of the range is a value less than the upper bound of the range.

The excitation signal can be applied to the graphene varactors at a frequency as dictated by the CDC or other hardware component. The frequency of the applied excitation signal can include a frequency that can be greater than or equal to 10 kHz, 20 kHz, 30 kHz, 40 kHz, 50 kHz, 60 kHz, 70 kHz, 80 kHz, 90 kHz, or 100 kHz, 125 kHz, 150 kHz, 175 kHz, 200 kHz, 225 kHz, 250 kHz, 275 kHz, 300 kHz, 325 kHz, 350 kHz, 375 kHz, 400 kHz, 425 kHz, 450 kHz, 475 kHz, 500 kHz, 525 kHz, 550 kHz, 575 kHz, 600 kHz, 625 kHz, 650 kHz, 675 kHz, 700 kHz, 725 kHz, 750 kHz, 775 kHz, 800 kHz, 825 kHz, 850 kHz, 875 kHz, 900 kHz, 925 kHz, 950 kHz, 975 kHz, or 1000 MHz or can be an amount falling within a range, wherein any of the foregoing frequencies can serve as the lower or upper bound of the range, provided that the lower bound of the range is a value less than the upper bound of the range.

In some embodiments, the total time for all measurement cycles can be configured to match the total amount of time for testing of a gaseous sample. In some embodiments, the total time for all measurement cycles can be configured to be equal to a predetermined time that covers a period of interest. In some embodiments, the total time for all measurement cycles can be configured to be equal or greater than the total amount of time for a non-steady state phase (or kinetic phase). In some embodiments, the controller circuit can be configured to determine the start of a non-steady state response phase from each of the discrete graphene components by assessing a rate of change of measured capacitance over time and initiate measurement cycles at that point. In some embodiments, the controller circuit can be configured to initiate measurement cycles when a signal is received indicating the start of a particular test of gaseous sample, such as receiving a sign from a flow sensor that a sample gas is starting to flow to the discrete graphene components. In some embodiments, the controller circuit can be configured to determine the end of a non-steady state phase by assessing a rate of change of measured capacitance over time and terminating measurement cycles at that point or reducing the frequency of measurement cycles at that point.

In various embodiments, the total time period for generating a series of measurement cycles (the total time for all measurement cycles) can include from 10 seconds to 1200 seconds. In some embodiments, the time period for generating a series of measurement cycles can include from 30 seconds to 180 seconds. In some embodiments, the time period for generating a series of measurement cycles can include from 10, 15, 20, 25, 30, 40, 45, 60, 90, 120, 150, 180, 360, 540, 720, 1080, 1200 seconds or more. It will be appreciated that the time period for generating a series of measurement cycles can include a range, wherein any of the forgoing time points can serve as the lower or upper bound of the range, provided that the lower bound of the range is a value less than the upper bound of the range.

In some embodiments, stepping through the range of applied voltages can include stepping through the range of applied voltages in predetermined increments, such as 50 mV increments. In some embodiments, stepping through the range of applied voltages can include stepping through the range of applied voltages in 10 mV increments. Stepping through the range of applied voltages can be performed at voltage increments of 1 mV, 5 mV, 10 mV, 25 mV, 50 mV, 75 mV, 100 mV, 125 mV, 150 mV, 200 mV, 300 mV, 400 mV, or 500 mV, or by a stepped amount falling within a range between any of the foregoing. In various embodiments, stepping through the range of applied voltages can include stepping through the range of applied voltages in increments from 1 mV to 500 mV. In various embodiments, stepping through the range of applied voltages can include stepping through the range of applied voltages in increments from 5 mV to 300 mV.

### Methods

Many different methods are contemplated herein, including, but not limited to, methods of making, methods of using, and the like. Aspects of system/device operation described elsewhere herein can be performed as operations of one or more methods in accordance with various embodiments herein.

In various embodiments, operations described herein and method steps can be performed as part of a computer-implemented method executed by one or more processors of one or more computing devices. In various embodiments, operations described herein and method steps can be implemented instructions stored on a non- transitory, computer-readable medium that, when executed by one or more processors, cause a system to execute the operations and/or steps.

In an embodiment, a method of detecting poisoning of a subject is included. The method can include taking a breath sample of the subject, taking measurements of the breath sample using a graphene sensor modified with one or more metalloporphyrins to generate sample data, and evaluating the sample data to detect a response associated with metalloporphyrin modified graphene.

In an embodiment, a method of detecting overdosed compounds in a subject is included. The method can include taking a breath sample of the subject, taking measurements of the breath sample using a graphene sensor modified with one or more metalloporphyrins and/or other modifications such as one or more acids to generate sample data, and evaluating the sample data to detect a response associated with the modified graphene.

In an embodiment of the method, the metalloporphyrins are in the form of a self-assembling monolayer. In an embodiment of the method, the metalloporphyrins include iron metalloporphyrins and/or cobalt metalloporphyrins. In an embodiment of the method, the metalloporphyrins include ruthenium metalloporphyrins.

In some embodiments, at least some graphene surfaces are modified with an acid. In an embodiment, the method can further include providing a voltage stimulus to the graphene sensor and measuring resulting capacitance values of the graphene sensor elements. In an embodiment, the method can further include providing a voltage stimulus to the graphene sensor over a range of voltage values and measuring resulting capacitance values of the graphene sensor elements.

In an embodiment of the method, the graphene sensor includes a graphene varactor.

In an embodiment of the method, the method is a method of detecting poisoning with a cyanide anion.

In an embodiment of the method, the method is a method for detecting poisoning with fentanyl or mustard gas.

In an embodiment of the method, the method is a method for detecting an overdose with fentanyl by itself and/or with other compounds present including ketamine and xylazine.

In an embodiment of the method, the response associated with metalloporphyrin modified graphene includes one or more of a change in the Dirac point upon a forward voltage sweep, a change in the Dirac point upon a reverse voltage sweep, a change in the capacitance of the Dirac point, and a change in the hysteresis between the forward and reverse Dirac points. In an embodiment of the method, the response associated with metalloporphyrin modified graphene includes a change in the minimum capacitance of the Dirac point. In an embodiment of the method, the response associated with metalloporphyrin modified graphene is relative to a non-metalloporphyrin modified graphene sensor.

Aspects may be better understood with reference to the following examples. These examples are intended to be representative of specific embodiments, but are not intended as limiting the overall scope of embodiments herein.

### EXAMPLES

### Example 1: Detection of Cyanide in a Porcine Model

A cyanide compound was administered to pigs intravenously and breath samples were taken immediately prior to administration and then again one minute after administration. The breath samples were evaluated with a system herein including graphene varactors with various compounds modifying surfaces thereof.

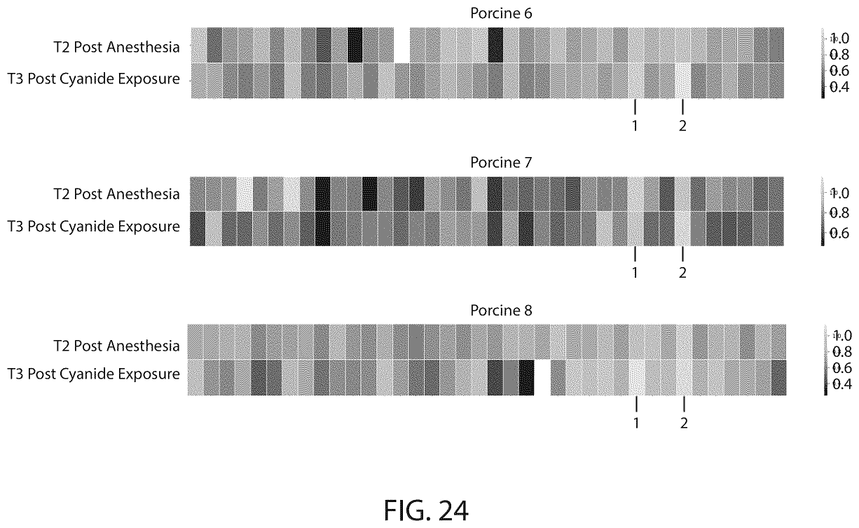

The results are shown in FIG. 24, which is a set of three charts (representing three different pigs) showing the response graphene varactors modified with various chemical compounds before and after exposure to a cyanide containing gas sample. In particular, “1” represents modification of a graphene surface with Co[II]C₁₂,C₁₂-porphryin and “2” represents modification of a graphene surface with Fe[III]C₁₂,C₁₂-porphryin. The top row of each chart shows the minimum capacitance of the Dirac point of 38 different sensor receptor surfaces just before the cyanide was given and the bottom row is the minimum capacitance values of the Dirac point one minute after cyanide was given. As can be seen, the response of functionalizations 1 and 2 showed consistent responses to the presence of cyanide in the porcine system. Receptor 1 is a cobalt metalloporphyrin and receptor 2 is an iron metalloporphyrin, both of which responded quickly to the breath content following cyanide administration. This example shows that cyanide poisoning can be detected using embodiments of systems herein including cobalt metalloporphyrins and/or iron metalloporphyrins.

### Example 2: Detection of Fentanyl and Fentanyl in Combination with Other Compounds in a Porcine Model

Pigs were sedated with ketamine or xylazine, anesthetized with isoflurane, and then administered a dose of fentanyl. Breath samples were taken at various time points as follows:
T-1—The animal is administered a sedative, ketamine or xylazine. Breath data was collected once the animal was sedated and laying quietly in the pen using a veterinary anesthesia mask.
T-2—After the pig was intubated and prepared for fentanyl exposure, isoflurane was administered through a ventilation system. Once the animal was stable, breath data collection occurred with minimal interference of the flow of air/anesthetic circuit.
T-3—Following ten seconds after the intravenous flow of fentanyl was initiated, breath data was collected for 1 minute, again testing the exhaled breath as it returned to the ventilation system
T-4—Eight minutes post fentanyl, a breath test was performed while the animal was on ventilation.

The breath samples were evaluated with a system herein including graphene varactors with various compounds modifying surfaces thereof.

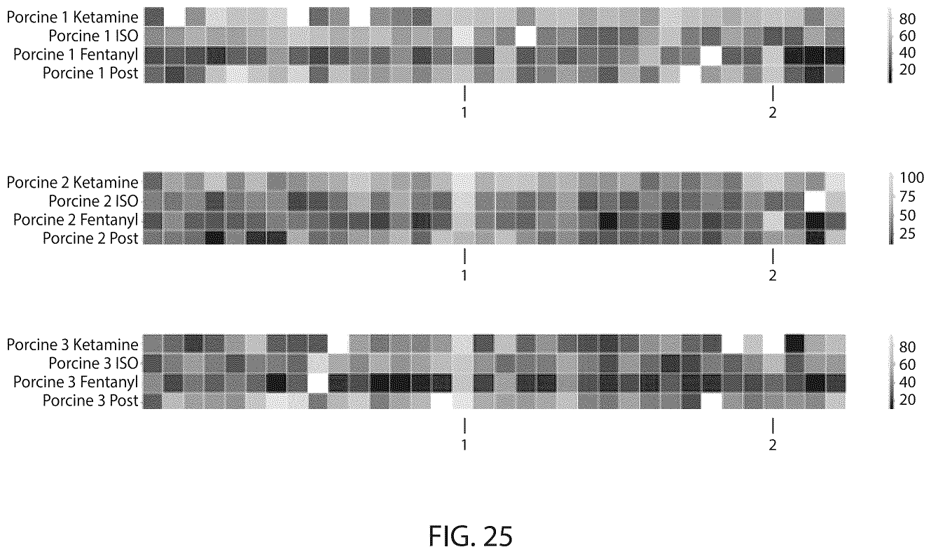

The results are shown in FIG. 25, which is a set of three charts (representing two pigs that received ketamine—top and bottom charts, and one pig that received xylazine) showing the response graphene varactors modified with various chemical compounds at the four time points discussed above (T-1 at the top, T-4 at the bottom). In particular, “1” represents modification of a graphene surface with pyrene-boronic acid and “2” represents modification of a graphene surface with Ru(II)C₁₂, C₁₂-porphyrin. The top row of each chart shows the minimum capacitance of the Dirac point of 38 different sensor receptor surfaces. As can be seen, the response of functionalizations 1 and 2 showed consistent responses to the presence of fentanyl in the porcine system. In addition, a response was shown to ketamine and xylazine.

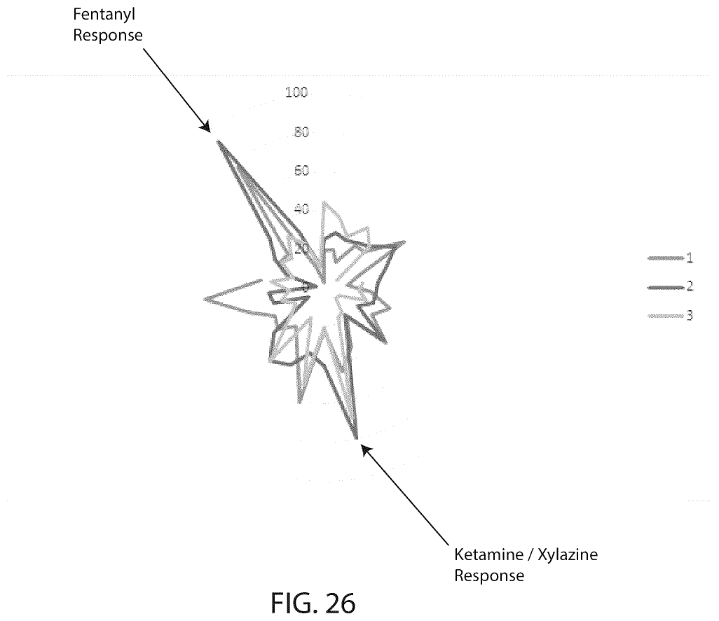

FIG. 26 is a radar plot showing the results of fentanyl exposure in combination with other compounds referenced above. As can be seen, the ketamine/xylazine response and the fentanyl response are both visible.

This example shows that overdosed compounds (such as fentanyl) can be detected using embodiments of systems herein. In addition, this example shows that the presence of multiple compounds can be detected simultaneously (such as fentanyl along with ketamine or xylazine), which can allow effective diagnosis and treatment of an overdose.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A system for detecting poisoning of a subject comprising:
a sensor device, the sensor device comprising a set of graphene sensor elements, wherein at least some of the set of graphene sensor elements are modified with metalloporphyrin compounds;
a measurement circuit;
wherein the system is configured to
receive a breath sample of the subject;
take measurements of the breath sample using the set of graphene sensor elements to generate sample data; and
evaluate the sample data to detect a pattern showing an enhanced response of the set of graphene sensor elements modified with metalloporphyrins indicating poisoning of the subject.

2. The system of claim 1, the metalloporphyrin compounds comprising cobalt metalloporphyrins.

3. The system of claim 1, the metalloporphyrin compounds comprising iron metalloporphyrins.

4. The system of claim 1, wherein the measurement circuit is configured to provide a voltage stimulus to the sensor device and measure resulting capacitance values of the graphene sensor elements.

5. (canceled)

6. The system of claim 1, the set of graphene sensor elements comprising graphene varactors.

7. The system of claim 1, wherein the system is a system for detecting poisoning with a cyanide anion.

8. The system of claim 1, wherein the system is a system for detecting poisoning with fentanyl or mustard gas.

9. The system of claim 1, wherein the enhanced response includes one or more of a change in the Dirac point upon a forward voltage sweep, a change in the Dirac point upon a reverse voltage sweep, a change in the capacitance of the Dirac point, and a change in the hysteresis between the forward and reverse Dirac points.

10-11. (canceled)

12. A system for detecting poisoning of a subject comprising:
a sensor device, the sensor device comprising
a set of graphene sensor elements, wherein one or more compounds are disposed on a surface of the set of graphene sensor elements;
the one or more compounds comprising metalloporphyrins;
a measurement circuit;
wherein the system is configured to
receive a breath sample of the subject;
take measurements of the breath sample using the set of graphene sensor elements to generate sample data; and
evaluate the sample data to detect an enhanced response of the set of graphene sensor elements with metalloporphyrins disposed thereon indicating poisoning of the subject.

13. The system of claim 12, the metalloporphyrins comprising cobalt metalloporphyrins.

14. The system of claim 12, the metalloporphyrins comprising iron metalloporphyrins.

15-17. (canceled)

18. The system of claim 12, the set of graphene sensor elements comprising graphene varactors.

19. The system of claim 12, wherein the system is a system for detecting poisoning with a cyanide anion.

20. The system of claim 12, wherein the system is a system for detecting poisoning with fentanyl or mustard gas.

21-33. (canceled)

34. A system for detecting poisoning of a subject comprising:
a sensor device, the sensor device comprising a set of graphene sensor elements, wherein at least some of the set of graphene sensor elements are modified with metalloporphyrin compounds;
a measurement circuit;
wherein the system is configured to
receive a breath sample of the subject;
take measurements of the breath sample using the set of graphene sensor elements to generate sample data; and
evaluate the sample data to detect a pattern showing an enhanced response of the set of graphene sensor elements modified with metalloporphyrins and/or one or more other modifying compounds indicating the presence of a particular compound or a particular combination of compounds in the subject.

35. The system of claim 34, the metalloporphyrin compounds comprising a ruthenium metalloporphyrin.

36-38. (canceled)

39. The system of claim 34, wherein the system is a system for detecting the presence of and/or overdose with fentanyl.

40. The system of claim 34, wherein the system is a system for detecting the presence of and/or overdose with fentanyl and another compound.

41. The system of claim 34, wherein the system is a system for distinguishing between overdose with fentanyl alone and overdose with fentanyl and another compound.

42. The system of claim 34, wherein the system is a system for distinguishing between overdose with fentanyl alone and overdose with fentanyl and ketamine and/or xylazine.

43-70. (canceled)
